(19) United States
(12) Patent Application Publication
(10) Pub. No.: US 2024/0271107 A1
(43) Pub. Date: Aug. 15, 2024

(54) VECTOR MANUFACTURING PROCESSES

(71) Applicant: 2SEVENTY BIO, INC., Cambridge, MA (US)

(72) Inventors: Kelly Marie Kral, Boston, MA (US); Michael Christopher Kuczewski, Newton, MA (US); Moumita Bhattacharya, Andover, MA (US); Kristen Kelly, Pembroke, MA (US); Adnan Kapadia, Boston, MA (US)

(21) Appl. No.: 18/580,298

(22) PCT Filed: Jul. 19, 2022
(86) PCT No.: PCT/US2022/037544

§ 371 Date: Jan. 18, 2024

Related U.S. Application Data
(60) Provisional application No. 63/223,249, filed on Jul. 19, 2021.

(51) Int. Cl. C12N 7/00; B01D 15/36; B01D 15/38; B01D 61/14; B01D 63/02; C12N 5/071; C12N 9/22; C12N 15/86

(52) U.S. Cl. CPC C12N 7/00; B01D 15/362; B01D 15/3823; B01D 61/145; B01D 63/02; C12N 5/0686; C12N 9/22; C12N 15/86; B01D 2315/16; C12N 2740/15052; C12N 2740/16051; C12N 2740/16052

(57) ABSTRACT

The present disclosure provides viral vector manufacturing and purification methods. Particularly, the disclosure provides improved methods for manufacturing lentiviral vector from host cells grown in suspension. More particularly, the disclosure provides improved large-scale lentiviral manufacturing methods comprising growing cells to a suitable number, transfecting with lentiviral packaging plasmids, a transfer plasmid, and a transfection agent; treating with an endonuclease; harvesting and clarifying the suspension culture supernatant; capturing and concentrating the lentiviral vector using affinity chromatography or cation exchange chromatography; filtering the concentrated lentiviral vector; ultrafiltering and diafiltering the lentiviral vector; formulating the lentiviral vector; and sterile filtering the formulated bulk lentiviral vector.

### CROSS REFERENCE TO RELATED APPLICATIONS

This application claims the benefit under 35 U.S.C. § 119(e) of U.S. Provisional Application No. 63/223,249, filed Jul. 19, 2021, which is incorporated by reference herein in its entirety.

### TECHNICAL FIELD

The present invention relates to improved methods of viral vector production and purification. The invention also relates to improved methods of manufacturing viral vectors.

### DESCRIPTION OF THE RELATED ART

Retroviral vectors of both oncoretroviral and lentiviral origins have a wealth of, as of yet, unmet potential as gene delivery vehicles. Large-scale manufacturing for clinical grade viral gene therapy vectors faces numerous obstacles including scalable unit operations for vector production and purification that removes contaminants while maintaining vector stability, titer and potency.

### BRIEF SUMMARY

The present disclosure generally relates, in part, to improved methods for manufacturing lentiviral vector. Particularly, the disclosure provides improved methods for manufacturing lentiviral vector from host cells grown in suspension.

In one aspect of the disclosure, a suspension process for producing lentiviral vector (sLVV) is provided, comprising: inoculating a large-scale suspension culture with viable host cells; transiently transfecting the host cells in the large-scale suspension culture with a mixture comprising lentiviral packaging plasmids, a transfer plasmid, and a transfection agent; adding an endonuclease to the suspension culture supernatant about 36 hours to about 48 hours post-transfection (after the initiation of transfection); harvesting and clarifying the suspension culture superatant using a tandem depth filter and a dual-layer filter; capturing and concentrating the lentiviral vector from the harvested and clarified suspension culture supernatant using chromatography; filtering the concentrated lentiviral vector; ultrafiltering and diafiltering the lentiviral vector using Tangential Flow Filtration (TFF); and formulating the lentiviral vector to produce a formulated bulk lentiviral vector, and sterile filtering the formulated bulk lentiviral vector.

In various embodiments, the process comprises inoculating a suspension culture of 200 L to 2000 L. In some embodiments, the process comprises inoculating a suspension culture of 200 L to 1000 L. In some embodiments, the process comprises inoculating a suspension culture of 200 L to 500 L. In some embodiments, the process comprises inoculating a suspension culture of 200 L.

In various embodiments, the large-scale suspension culture is inoculated with about 40.0×10⁸ to about 120.0×10⁸ viable host cells. In some embodiments, the host cells are selected from the group consisting of HEK293 cells, HEK293S cells, HEK293T cells adapted for suspension culture (HEK293Ts), HEK293F cells, HEK293FT cells, HEK293FTM cells, and HEK293E cells. In some embodiments, the host cells are HEK293Ts cells.

In various embodiments, the large-scale cell suspension culture comprises host cells cultured in a culture medium. In some embodiments, the large-scale cell suspension culture comprises host cells cultured in a culture medium for about 3 days, wherein after the 3 days, the culture medium is exchanged for fresh culture medium. In some embodiments, the large-scale cell suspension culture comprises host cells cultured in a culture medium for about 3 days, wherein after the 3 days, the culture medium is exchanged for fresh culture medium using Alternating Tangential Flow Filtration (ATF). In some embodiments, the culture medium is a serum-free chemically defined cell culture medium.

In various embodiments, the host cells are transiently transfected with the mixture comprising a transfection agent selected from the group consisting of: calcium phosphate, cationic lipids, and cationic polymers. In some embodiments, the host cells are transiently transfected with the mixture comprising a transfection agent that is a cationic polymer selected from the group consisting of: DEAE-dextran, poly brene, dendrimers, and polyethylenimine (PEI). In some embodiments, the host cells are transiently transfected with the mixture comprising a transfection agent that comprises PEI. In some embodiments, the transfection agent comprises PEI and the mixture has a ratio of N (NH2 amines in PEI):P (phosphate groups in DNA backbone) of about 5, about 5.5, about 6, about 6.4, about 6.5, about 7, about 7.5, about 8, about 8.5, about 9, about 9.5, or about 10.

In various embodiments, the transfection agent is added to the suspension culture for about 14 to about 18 hours, optionally wherein the suspension culture is subjected to a culture medium exchange with fresh culture medium using Alternating Tangential Flow Filtration (ATF).

In various embodiments, the lentiviral packaging plasmids encode lentiviral gag, pol, and rev and a heterologous envelope protein.

In various embodiments, the lentiviral packaging plasmids encode a heterologous envelope protein selected from the group consisting of a Vesiculovirus envelope protein or variant thereof, Paramyxoviridae envelope protein or variant thereof, an Alphavirus envelope protein or variant thereof, a Gammaretrovirus envelope protein or variant thereof, an Orthohepadnavirus envelope protein or variant thereof, a Hepacivirus envelope protein or variant thereof, and a Lyssavirus envelope protein or variant thereof.

In particular embodiments, the lentiviral packaging plasmids encode a heterologous envelope protein selected from the group consisting of a Nipah virus envelope protein, a Sendai virus (SeV) envelope protein, a Morbillivirus envelope protein, a Canine distemper (CDV) envelope protein, and a Measles virus envelope protein.

In various embodiments, the lentiviral packaging plasmids encode a Sindbis virus (SINV) envelope protein.

In various embodiments, the lentiviral packaging plasmids encode a heterologous envelope protein selected from the group consisting of a Gibbon ape leukemia virus (GALV) envelope protein, a Feline leukemia virus (FeLV) envelope protein, a Feline endogenous retrovirus (RD114) envelope protein, and a Baboon endogenous retrovirus (BaEV) envelope protein

In various embodiments, the lentiviral packaging plasmids encode a Hepatitis B virus (HBV) envelope protein.

In various embodiments, the lentiviral packaging plasmids encode a Hepatitis C virus (HCV) envelope protein.

In various embodiments, the lentiviral packaging plasmids encode a Rabis virus (RABV).

In various embodiments, the lentiviral packaging plasmids encode a Vesicular stomatitis virus (VSV) envelope protein or variant thereof (e.g., VSV-G), a Cocal virus (COCV) envelope protein or variant thereof, a Maraba virus (MARAV) envelope protein or variant thereof, a Piry virus (PIRYV) envelope protein or variant thereof, a Nipah virus (NiV) envelope protein or variant thereof, a Sendai virus (SeV) envelope protein or variant thereof, a Morbillivirus envelope protein or variant thereof, a Canine distemper (CDV) envelope protein or variant thereof, a Measles virus (MV) envelope protein or variant thereof, a Sindbis virus (SINV) envelope protein or variant thereof, a Gibbon ape leukemia virus (GALV) envelope protein or variant thereof, a Feline endogenous retrovirus (RD114) envelope protein or variant thereof, a Feline leukemia virus (FeLV) envelope protein or variant thereof, a Baboon endogenous retrovirus (BaEV) envelope protein or variant thereof, a Hepatitis B (HBV) envelope protein or variant thereof, a Hepatitis C (HCV) envelope protein or variant thereof, and a Rabis virus (RABV) envelope protein or variant thereof.

In a preferred embodiment, the VSV envelope protein is a VSV-G or variant thereof.

In various embodiments, the transfer plasmid comprises a polynucleotide comprising a packageable lentiviral vector genome. In some embodiments, the transfer plasmid comprises a polynucleotide comprising a packageable lentiviral vector genome comprising a left chimeric (5′) lentiviral LTR, the promoter of the 5′ LTR is replaced with a heterologous promoter; a Psi (Ψ) packaging signal; a central polypurine tract/DNA flap (cPPT/FLAP); a retroviral export element (RRE); a promoter operably linked to a polynucleotide of interest; and a right (3′) self-inactivating (SIN) lentiviral LTR.

In various embodiments, the endonuclease is derived from Serratia marcescens, optionally wherein the endonuclease is a recombinant NucA endonuclease. In some embodiments, the endonuclease has both DNA and RNA cleaving activity. In some embodiments, the endonuclease is Benzonase or Denarase. In some embodiments, the endonuclease is added at a concentration of about 60 U/ml or about 30 U/ml.

In various embodiments, the endonuclease is added to the suspension culture supernatant about 36 hours to about 72 hours post-transfection. In some embodiments, the endonuclease is added to the suspension culture supernatant about 36 hours to about 48 hours post-transfection. In some embodiments, the endonuclease is added to the suspension culture supernatant about 48 hours post-transfection. In some embodiments, the endonuclease is added to the suspension culture supernatant about 44 hours post-transfection. In some embodiments, the endonuclease is added to the suspension culture supernatant about 40 hours after transfection. In some embodiments, the endonuclease is added to the suspension culture supernatant about 36 hours after transfection.

In various embodiments, the endonuclease is added to the culture for about 1 to about 2 hours.

In various embodiments, the harvesting and clarifying step comprises filtering the suspension culture supernatant through the tandem depth filter retains contaminants of at least about 40 μm or at least about 60 μm and a dual-layer filter comprising a pre-filter pore size of about 0.45 μm to about 0.8 μm and a final filter pore size of about 0.22 μm to about 0.45 μm.

In various embodiments, the clarified suspension culture is adjusted to about pH 7.0 or about pH 7.2, optionally with 1M HEPES.

In various embodiments, the lentiviral vector is captured and concentrated from the harvested and clarified suspension culture supernatant using affinity chromatography or cation exchange chromatography. In some embodiments, the supernatant is passed over an affinity chromatography column or cation exchange chromatography column. In some embodiments, the affinity chromatography is a heparin affinity chromatography. In some embodiments, the cation exchange chromatography is a sulfate cation exchange chromatography. In some embodiments, the sulfate cation exchange chromatography comprises a column having bead size of about 45 μm and/or a mean pore size of about 100 nm.

In various embodiments, a wash buffer comprising about 50 mM HEPES, about 100 mM NaCl, pH 7 is pumped over the chromatography column.

In various embodiments, an elution buffer comprising about 50 mM HEPES, about 400 mM NaCl, pH 8 is pumped over the chromatography column.

In various embodiments, a wash buffer comprising about 50 mM HEPES, about 300 mM NaCl, pH 7.2 is passed over the chromatography column. In various embodiments, an elution buffer comprising about 50 mM HEPES, about 1 M NaCl, pH 7.5 is passed over the chromatography column.

In various embodiments, the filtering of the concentrated vector comprises filtering the concentrated lentiviral vector through a dual-layer filter comprising a pre-filter pore size of about 0.45 μm to about 0.8 μm and a final filter pore size of about 0.2 μm to about 0.45 μm.

In various embodiments, the lentiviral vector is ultrafiltered and diafiltered using a hollow fiber Tangential Flow Filtration (TFF) filter comprising an about 100 kDa to about 500 kDa pore size or molecular weight cutoff. In some embodiments, the hollow fiber TFF filter comprises a pore size or molecular weight cutoff of about 100 kDa. In some embodiments, the hollow fiber TFF filter comprises a pore size or molecular weight cutoff of about 300 kDa. In some embodiments, the hollow fiber TFF filter comprises a pore size or molecular weight cutoff of about 500 kDa.

In various embodiments, the lentiviral vector is diafiltered into diafiltration buffer, optionally wherein the diafiltration buffer is about 50 mM HEPES, about 100 mM NaCl, pH 7.50.

In various embodiments, the lentiviral vector is diafiltered into diafiltration buffer, optionally wherein the diafiltration buffer is about 50 mM HEPES, pH 7.0.

In various embodiments, the lentiviral vector is diafiltered into diafiltration buffer, optionally wherein the diafiltration buffer is about 50 mM L-Histidine, pH 7.0.

In various embodiments, the lentiviral vector is formulated 1:1 in 2× Stem Cell Growth Medium (SCGM) to produce the formulated bulk lentiviral vector.

In various embodiments, the lentiviral vector is formulated 1:1 in a buffer comprising HEPES and Sucrose, optionally wherein the buffer further comprises L-proline, poloxamer 188, or NaCl.

In various embodiments, the lentiviral vector is formulated 1:1 in a buffer comprising about 5 mM HEPES (pH 7.0), about 146 mM Sucrose, and about 100 mM L-proline.

In various embodiments, the lentiviral vector is formulated 1:1 in a buffer comprising about 5 mM HEPES (pH 7.0), about 146 mM Sucrose, about 100 mM L-proline, and about 0.2 to about 2.0 mg/ml poloxamer 188.

In various embodiments, the lentiviral vector is formulated 1:1 in a buffer comprising about 5 mM HEPES (pH 7.0), about 146 mM Sucrose, about 100 mM L-proline, and about 150 mM NaCl.

In various embodiments, the lentiviral vector is formulated 1:1 in a buffer comprising about 5 mM HEPES (pH 7.0), about 146 mM Sucrose, about 100 mM L-proline, about 150 mM NaCl, and about 0.2 to about 2.0 mg/ml poloxamer 188.

In various embodiments, the lentiviral vector is formulated 1:1 in a buffer comprising about 5 mM HEPES (pH 7.0), about 146 mM Sucrose, about 150 mM NaCl, and about 0.2 to about 2.0 mg/ml poloxamer 188.

In various embodiments, the lentiviral vector is formulated 1:1 in a buffer comprising L-Histidine, Sucrose, and L-proline.

In various embodiments, the lentiviral vector is formulated 1:1 in a buffer comprising about 5 mM L-Histidine, about 146 mM Sucrose, and about 100 mM L-proline, optionally wherein the formulation further comprises about 0.2 to about 2.0 mg/mL poloxamer 188.

In various embodiments, the formulated bulk lentiviral vector is sterile filtered through a 0.22 μm filter, optionally comprising a 0.45 μm pre-filter.

In various embodiments, the process further comprises performing a fill finish of the formulated bulk lentiviral vector to produce a final lentiviral vector, and freezing the final lentiviral vector.

In various embodiments, the process further comprises freezing the formulated bulk lentiviral vector.

In various embodiments, the process further comprises thawing the formulated bulk lentiviral vector, sterile filtering the formulated bulk lentiviral vector, performing a fill finish of the formulated bulk lentiviral vector to produce a final lentiviral vector, and freezing the final lentiviral vector. In some embodiments, the final lentiviral vector is frozen at ≤−65° C.

In another aspect, a method for producing suspension lentiviral vector is provided, comprising: inoculating a P0 suspension culture comprising about 50 mL of culture medium with about 10.0×10⁶ to about 15.0×10⁶ viable HEK293Ts cells; inoculating a P1 suspension culture comprising about 100 mL of culture medium with about 30.0×10⁶ to about 70.0×10⁶ viable HEK293Ts cells obtained from the P0 suspension culture; inoculating three P2 suspension cultures each comprising about 200 mL of culture medium with about 11.0×10⁷ to about 19.0×10⁷ viable HEK293Ts cells obtained from the P1 suspension culture; inoculating three P3 suspension cultures each comprising about 1.0 L of culture medium with about 55.0×10⁷ to about 95.0×10⁷ viable HEK293Ts cells obtained from the pooled P2 suspension cultures; inoculating a P4 suspension culture comprising about 20.0 L of culture medium with about 40.0×10⁸ to about 120.0×10⁸ viable HEK293Ts cells obtained from the pooled P3 suspension cultures; inoculating a P5 suspension culture comprising about 200.0 L of culture medium with about 40.0×10⁸ to about 120.0×10⁸ viable HEK293Ts cells obtained from the P4 suspension culture; culturing the P5 suspension culture for about 3 days and exchanging the culture medium of the P5 suspension culture with about 190.0 L of fresh culture medium using Alternating Tangential Flow Filtration (ATF); transfecting the P5 suspension culture, after the culture medium exchange, to produce a lentiviral vector, said transfecting step comprising adding about 10.0 L of culture medium comprising a transfer plasmid and plasmid DNAs encoding, gag, pol, rev, VSV-g, complexed with polyethyleneimine (PEI); exchanging the culture medium of the P5 suspension culture, post-transfection, with about 200.0 L of fresh culture medium using ATF; about 36 hours to about 48 hours after post-transfection, treating the P5 suspension culture with an endonuclease for about 1 hour to about 2 hours; harvesting and clarifying the P5 suspension culture supernatant using a tandem depth filter that retains particles of 60 μm or greater, and a dual-layer filter comprising 0.8 μm and 0.45 μm pore sizes; capturing and concentrating the lentiviral vector from the harvested and clarified P5 suspension culture supernatant comprising heparin chromatography or sulfate cation exchange chromatography; filtering the concentrated lentiviral vector using a dual-layer filter comprising 0.8 μm and 0.45 μm pore sizes; ultrafiltering the lentiviral vector using Tangential Flow Filtration (TFF) to further concentrate the lentiviral vector and diafiltering the lentiviral vector into diafiltration buffer, thereby producing bulk lentiviral vector; and formulating the lentiviral vector to produce a formulated bulk lentiviral vector.

In various embodiments, the culture medium is a serum-free chemically defined cell culture medium.

In various embodiments, the ratio of N (NH2 amines in PEI):P (phosphate groups in DNA backbone) for the PEI/DNA mixture is about 5, about 5.5, about 6, about 6.4, about 6.5, about 7, about 7.5, about 8, about 8.5, about 9, about 9.5, or about 10.

In various embodiments, the PEI is added to the suspension culture for about 14 to about 18 hours, optionally wherein the suspension culture is subjected to a culture medium exchange with fresh culture medium using Alternating Tangential Flow Filtration (ATF).

In various embodiments, the transfer plasmid comprises a polynucleotide comprising a packageable lentiviral vector genome. In some embodiments, the transfer plasmid comprises a polynucleotide comprising a packageable lentiviral vector genome comprising a left chimeric (5′) lentiviral LTR, the promoter of the 5′ LTR is replaced with a heterologous promoter; a Psi (Ψ) packaging signal; a central polypurine tract/DNA flap (cPPT/FLAP); a retroviral export element (RRE); a promoter operably linked to a polynucleotide of interest; and a right (3′) self-inactivating (SIN) lentiviral LTR.

In various embodiments, the endonuclease is derived from Serratia marcescens, optionally wherein the endonuclease is a recombinant NucA endonuclease. In some embodiments, the endonuclease has both DNA and RNA cleaving activity. In some embodiments, the endonuclease is Benzonase or Denarase. In some embodiments, the endonuclease is added at a concentration of about 60 U/ml or about 30 U/ml

In various embodiments, the clarified suspension culture is adjusted to about pH 7.0 or about pH 7.2, optionally with 1M HEPES.

In various embodiments, the sulfate cation exchange chromatography comprises a column having bead size of about 45 μm and/or a mean pore size of about 100 nm.

In various embodiments, a wash buffer comprising about 50 mM HEPES, about 300 mM NaCl, pH 7.2 is pumped over the affinity chromatography column or cation exchange chromatography column.

In various embodiments, an elution buffer comprising about 50 mM HEPES, about 1 M NaCl, pH 7.5 is pumped over the affinity chromatography column or cation exchange chromatography.

In various embodiments, the lentiviral vector is ultrafiltered and diafiltered into diafiltration buffer using a hollow fiber TFF filter comprising a pore size or molecular weight cutoff of about 100 kDa, about 300 kDa or about 500 kDa.

In various embodiments, the diafiltration buffer is about 50 mM HEPES, about 100 mM NaCl, pH 7.50.

In various embodiments, the diafiltration buffer is about 50 mM HEPES, pH 7.0.

In various embodiments, the lentiviral vector is formulated 1:1 in 2× Stem Cell Growth Medium (SCGM) to produce the formulated bulk lentiviral vector.

In various embodiments, the lentiviral vector is formulated 1:1 in a buffer comprising HEPES and Sucrose, optionally wherein the buffer further comprises L-proline, poloxamer 188, or NaCl.

In various embodiments, the lentiviral vector is formulated 1:1 in a buffer comprising about 5 mM HEPES (pH 7.0), about 146 mM Sucrose, and about 100 mM L-proline.

In various embodiments, the lentiviral vector is formulated 1:1 in a buffer comprising about 5 mM HEPES (pH 7.0), about 146 mM Sucrose, about 100 mM L-proline, and 0.2 mg/ml poloxamer 188.

In various embodiments, the lentiviral vector is formulated 1:1 in a buffer comprising about 5 mM HEPES (pH 7.0), about 146 mM Sucrose, about 100 mM L-proline, and about 150 mM NaCl.

In various embodiments, the lentiviral vector is formulated 1:1 in a buffer comprising about 5 mM HEPES (pH 7.0), about 146 mM Sucrose, about 100 mM L-proline, about 150 mM NaCl, and 0.2 mg/ml poloxamer 188.

In various embodiments, the lentiviral vector is formulated 1:1 in a buffer comprising about 5 mM HEPES (pH 7.0), about 146 mM Sucrose, about 150 mM NaCl, and 0.2 mg/ml poloxamer 188.

In various embodiments, the method further comprises performing a fill finish of the formulated bulk lentiviral vector to produce a final lentiviral vector, and freezing the final lentiviral vector.

In various embodiments, the method further comprises freezing the formulated bulk lentiviral vector.

In various embodiments, the process further comprises thawing the formulated bulk lentiviral vector, sterile filtering the formulated bulk lentiviral vector, performing a fill finish of the formulated bulk lentiviral vector to produce a final lentiviral vector, and freezing the final lentiviral vector. In some embodiments, the final lentiviral vector is frozen at ≤−65° C.

In another aspect, a method of reducing host cell protein (HCP) from a suspension process for producing viral vector is provided. In various embodiments the method for reducing HCP comprises: (a) providing a harvested and clarified suspension culture supernatant comprising viral vector (e.g., a manufactured viral vector contemplated herein); (b) capturing and concentrating the viral vector from the harvested and clarified suspension culture supernatant using cation exchange chromatography; (c) filtering the concentrated viral vector; (d) ultrafiltering and diafiltering the viral vector using Tangential Flow Filtration (TFF); and (e) formulating the viral vector to produce a formulated bulk viral vector, and sterile filtering the formulated bulk viral vector.

In various embodiments, the viral vector is a lentiviral vector.

In various embodiments, the viral vector is pseudotyped with a heterologous envelope protein selected from the group consisting of a Vesicular stomatitis virus (VSV) envelope protein or variant thereof (e.g., VSV-G), a Cocal virus (COCV) envelope protein or variant thereof, a Maraba virus (MARAV) envelope protein or variant thereof, a Piry virus (PIRYV) envelope protein or variant thereof, a Nipah virus (NiV) envelope protein or variant thereof, a Sendai virus (SeV) envelope protein or variant thereof, a Morbillivirus envelope protein or variant thereof, a Canine distemper (CDV) envelope protein or variant thereof, a Measles virus (MV) envelope protein or variant thereof, a Sindbis virus (SINV) envelope protein or variant thereof, a Gibbon ape leukemia virus (GALV) envelope protein or variant thereof, a Feline endogenous retrovirus (RD114) envelope protein or variant thereof, a Feline leukemia virus (FeLV) envelope protein or variant thereof, a Baboon endogenous retrovirus (BaEV) envelope protein or variant thereof, a Hepatitis B (HBV) envelope protein or variant thereof, a Hepatitis C (HCV) envelope protein or variant thereof, and a Rabis virus (RABV) envelope protein or variant thereof. In some embodiments, the viral vector is pseudotyped with a heterologous envelope protein consisting of a Vesicular stomatitis virus (VSV) envelope protein or variant thereof (e.g., VSV-G).

In various embodiments, the harvesting and clarifying step comprises filtering the suspension culture supernatant through the tandem depth filter retains contaminants of at least about 40 μm or at least about 60 μm and a dual-layer filter comprising a pre-filter pore size of about 0.45 μm to about 0.8 μm and a final filter pore size of about 0.22 μm to about 0.45 μm.

In various embodiments, the clarified suspension culture is adjusted to about pH 7.0 or about pH 7.2, optionally with 1M HEPES.

In various embodiments, the supernatant is passed over the cation exchange chromatography column.

In various embodiments, the cation exchange chromatography is a sulfate cation exchange chromatography. In some embodiments, the sulfate cation exchange chromatography comprises a column having bead size of about 45 μm and/or a mean pore size of about 100 nm.

In various embodiments, a wash buffer comprising about 50 mM HEPES, about 300 mM NaCl, pH 7.2 is passed over the chromatography column.

In various embodiments, an elution buffer comprising about 50 mM HEPES, about 1 M NaCl, pH 7.5 is passed over the chromatography column.

In various embodiments, the filtering step (c) comprises filtering the concentrated viral vector through a dual-layer filter comprising a pre-filter pore size of about 0.45 μm to about 0.8 μm and a final filter pore size of about 0.2 μm to about 0.45 μm.

In various embodiments, the viral vector is ultrafiltered and diafiltered using a hollow fiber Tangential Flow Filtration (TFF) filter comprising an about 100 kDa to about 500 kDa pore size or molecular weight cutoff. In some embodiments, the hollow fiber TFF filter comprises a pore size or molecular weight cutoff of about 100 kDa. In some embodiments, the hollow fiber TFF filter comprises a pore size or molecular weight cutoff of about 300 kDa. In some embodiments, the hollow fiber TFF filter comprises a pore size or molecular weight cutoff of about 500 kDa.

In various embodiments, the viral vector is diafiltered into diafiltration buffer, optionally wherein the diafiltration buffer is about 50 mM HEPES, pH 7.0.

In various embodiments, viral vector is formulated 1:1 in a buffer comprising HEPES and Sucrose, optionally wherein the buffer further comprises L-proline, poloxamer 188, or NaCl. In some embodiments, the viral vector is formulated 1:1 in a buffer comprising about 5 mM HEPES (pH 7.0), about 146 mM Sucrose, and about 100 mM L-proline. In some embodiments, the viral vector is formulated 1:1 in a buffer comprising about 5 mM HEPES (pH 7.0), about 146 mM Sucrose, about 100 mM L-proline, and about 0.2 to about 2.0 poloxamer 188. In some embodiments, the viral vector is formulated 1:1 in a buffer comprising about 5 mM HEPES (pH 7.0), about 146 mM Sucrose, about 100 mM L-proline, and about 150 mM NaCl. In some embodiments, the viral vector is formulated 1:1 in a buffer comprising about 5 mM HEPES (pH 7.0), about 146 mM Sucrose, about 100 mM L-proline, about 150 mM NaCl, and about 0.2 to about 2.0 mg/ml poloxamer 188. In some embodiments, the viral vector is formulated 1:1 in a buffer comprising about 5 mM HEPES (pH 7.0), about 146 mM Sucrose, about 150 mM NaCl, and about 0.2 to about 2.0 mg/ml poloxamer 188.

In various embodiments, the formulated bulk viral vector is sterile filtered through a 0.22 μm filter, optionally comprising a 0.45 μm pre-filter.

In various embodiments, the process further comprises performing a fill finish of the formulated bulk viral vector to produce a final viral vector, and freezing the final viral vector.

In various embodiments, the process further comprises freezing the formulated bulk viral vector. In some embodiments, the process further comprises thawing the formulated bulk viral vector, sterile filtering the formulated bulk viral vector, performing a fill finish of the formulated bulk viral vector to produce a final viral vector, and freezing the final viral vector.

In various embodiments, the final viral vector is frozen at ≤−65° C.

### BRIEF DESCRIPTION OF SEVERAL VIEWS OF THE DRAWINGS

FIG. 1 shows an example of an upstream process flow, a downstream process flow and a fill-finish for the production of lentiviral vector from suspension culture.

FIG. 2 shows an example of an upstream process flow, a downstream process flow and a fill-finish for the production of lentiviral vector from suspension culture.

FIGS. 3A-3C show the plasmid maps for packaging plasmids for gag, pol, rev, and vsv-g.

FIG. 4 shows an example of an upstream process flow, a downstream process flow and a fill-finish for the production of lentiviral vector from suspension culture.

FIG. 5 shows a comparison of total infectious titer yield between different LVV manufacturing methods.

FIG. 6 shows a comparison of infectious titer between different LVV manufacturing methods.

FIG. 7 shows a comparison of particle to infectivity ratio between different LVV manufacturing methods.

FIG. 8 shows a comparison of normalized host cell protein (HCP) between different LVV manufacturing methods.

FIG. 9 shows a comparison of cumulative host cell protein (HCP) log reduction between different LVV manufacturing methods.

FIG. 10 shows a comparison of step host cell protein (HCP) log reduction between different LVV manufacturing methods.

### DETAILED DESCRIPTION

### A. Overview

The present disclosure generally relates to, in part, improved large-scale processes for manufacturing retroviral vectors for clinical use. Without wishing to be bound by any particular theory, the present disclosure is the first to provide manufacturing processes to produce clinical grade retroviral and lentiviral vectors. Vectors produced using the manufacturing processes contemplated herein are produced at a clinical scale with infectivity and purity unmatched by methods existing in the art. Additionally, the present disclosure provides methods for reducing host cell protein (HCP) from manufactured viral vector superatants.

Manufacturing processes that exist in the art usually err on the side of increasing viral vector quantity at the expense of vector quality. The manufacturing processes contemplated herein solve the problems in the art of trading quantity for quality and enable the production of high-quality clinical grade vector at commercial scale. Indeed, the processes contemplated herein demonstrate higher infectious titer (TU/ml), and in some embodiments equivalent or lower host cell protein (HCP) levels.

The manufacturing processes contemplated comprise an upstream process that produces the viral vector and a downstream process that purifies the viral vector. The contemplated manufacturing processes comprise establishing large-scale host cell cultures in a bioreactor; transiently transfecting the host cells with mixture comprising packaging plasmids encoding viral accessory genes and a transfer plasmid; culturing transfected host cells to produce virus; and collecting and processing the culture supernatant that contains the crude lentiviral vector to remove impurities and concentrate and formulate the viral vector for clinical use.

In particular embodiments, manufacturing processes contemplated herein comprise an upstream process comprising thawing and culturing and expanding host cells in progressively larger volumes until a sufficient amount of host cells to seed a large-scale, e.g., at least 200 L working volume, bioreactor. The seeded host cells are cultured to a desired density, the medium exchanged, and the cells transfected with a mixture comprising a transfection agent, packaging plasmids encoding viral accessory genes and a transfer plasmid encoding a packageable viral vector genome comprising a therapeutic transgene. After a sufficient time for transfection, another medium exchange is performed and the transfected host cells are cultured to produce viral vector for about one to about three days. In preferred embodiments, the host cells are cultured in serum free chemically defined cell culture medium.

In particular embodiments, manufacturing processes contemplated herein comprise a downstream process comprising treating the contents of the bioreactor with a DNA endonuclease; harvesting and clarifying the suspension culture supernatant by filtration; capturing and concentrating the viral vector in the resultant filtrate using affinity chromatography or cation exchange chromatography; filtering the eluate comprising the viral vector; ultrafiltering and diafiltering the viral vector using tangential flow filtration (TFF); and formulating the viral vector in a culture medium to produce a formulated bulk viral vector. In one embodiment, the formulated bulk lentiviral vector is sterile filtered, filled, and frozen; and subsequently thawed, sterile filtered, subjected to a final fill finish, and frozen. In another embodiment, the bulk lentiviral vector is sterile filtered, subjected to a final fill finish, and frozen.

In preferred embodiments, the viral vector is a retroviral vector and in even more preferred embodiments, the viral vector is a lentiviral vector.

Techniques for recombinant (i.e., engineered) DNA, peptide and oligonucleotide synthesis, immunoassays, tissue culture, transformation (e.g., electroporation, lipofection), enzymatic reactions, purification and related techniques and procedures may be generally performed as described in various general and more specific references in microbiology. molecular biology, biochemistry, molecular genetics, cell biology, virology and immunology as cited and discussed throughout the present specification. See, e.g., Sambrook et al., Molecular Cloning: A Laboratory Manual, 3d ed., Cold Spring Harbor Laboratory Press, Cold Spring Harbor, N.Y.; Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford Univ. Press USA, 1985); Current Protocols in Immunology (Edited by: John E. Coligan, Ada M. Kruisbeek, David H. Margulies, Ethan M. Shevach, Warren Strober 2001 John Wiley & Sons, NY, NY); Real-Time PCR: Current Technology and Applications, Edited by Julie Logan, Kirstin Edwards and Nick Saunders, 2009, Caister Academic Press, Norfolk, UK; Anand, Techniques for the Analysis of Complex Genomes, (Academic Press, New York, 1992); Guthrie and Fink, Guide to Yeast Genetics and Molecular Biology (Academic Press, New York, 1991); Oligonucleotide Synthesis (N. Gait, Ed., 1984); Nucleic Acid The Hybridization (B. Hames & S. Higgins, Eds., 1985); Transcription and Translation (B. Hames & S. Higgins, Eds., 1984); Animal Cell Culture (R. Freshney, Ed., 1986); Perbal, A Practical Guide to Molecular Cloning (1984); Next-Generation Genome Sequencing (Janitz, 2008 Wiley-VCH); PCR Protocols (Methods in Molecular Biology) (Park, Ed., 3rd Edition, 2010 Humana Press); Immobilized Cells And Enzymes (IRL Press, 1986); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Harlow and Lane, Antibodies, (Cold Spring Harbor Laboratory Press, Cold Spring Harbor, N.Y., 1998); Immunochemical Methods In Cell And Molecular Biology (Mayer and Walker, eds., Academic Press, London, 1987); Handbook Of Experimental Immunology, Volumes I-IV (D. M. Weir and C C Blackwell, eds., 1986); Roitt, Essential Immunology, 6th Edition, (Blackwell Scientific Publications, Oxford, 1988); Current Protocols in Immunology (Q. E. Coligan, A. M. Kruisbeek, D. H. Margulies, E. M. Shevach and W. Strober, eds., 1991); Annual Review of Immunology; as well as monographs in journals such as Advances in Immunology.

### B. Definitions

Prior to setting forth this disclosure in more detail, it may be helpful to an understanding thereof to provide definitions of certain terms to be used herein.

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below.

The articles “a,” “an,” and “the” are used herein to refer to one or to more than one (i.e., to at least one, or to one or more) of the grammatical object of the article. By way of example, “an element” means one element or one or more elements.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

In one embodiment, a range, e.g., 1 to 5, about 1 to 5, or about 1 to about 5, refers to each numerical value encompassed by the range. For example, in one non-limiting and merely illustrative embodiment, the range “1 to 5” is equivalent to the expression 1, 2, 3, 4, 5; or 1.0, 1.5, 2.0, 2.5, 3.0, 3.5, 4.0, 4.5, or 5.0; or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher compared to a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are present that materially affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

The term “vector” is used herein to refer to a nucleic acid molecule, mircroorganism, or virus capable transferring or transporting another nucleic acid molecule to a cell or genome. Illustrative examples of vectors include, for example, plasmids (e.g., DNA plasmids or RNA plasmids), transposons, cosmids, bacterial artificial chromosomes, bacteria, and viral vectors.

The term “viral vector” is used in particular embodiments, to refer either to a nucleic acid molecule that includes virus-derived nucleic acid elements that typically facilitate transfer of the nucleic acid molecule or integration into a cell and/or genome. The term “viral vector” also refers to, in particular preferred embodiments, either to a modified virus or viral particle capable of transferring a nucleic acid into a cell and/or genome. Viral vectors may contain structural and/or functional genetic elements that are primarily derived from a virus. Viral vectors suitable for use in preferred embodiments, include but are not limited to retroviral vectors and lentiviral vectors.

Retroviral vectors are a common tool for gene delivery (Miller, 2000, Nature. 357: 455-460). As used herein, the term “retrovirus” or “retroviral vector” refers to a viral vector that reverse transcribes its genomic RNA into a linear double-stranded DNA copy and subsequently covalently integrates its genomic DNA into a host genome. Illustrative retroviral vectors suitable for use in particular embodiments, include, but are not limited to those derived from Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

As used herein, the term “lentivirus” refers to a group (or species) of complex retroviruses. Illustrative lentiviral vectors suitable for use in particular embodiments contemplated herein include, but are not limited to those derived from HIV (human immunodeficiency virus; including HIV type 1, and HIV type 2); visna-maedi virus (VMV); the caprine arthritis-encephalitis virus (CAEV); equine infectious anemia virus (EIAV); feline immunodeficiency virus (FIV); bovine immune deficiency virus (BIV); and simian immunodeficiency virus (SIV).

The term “provector” or “provirus” refers to a viral vector that has integrated into a host genome. Provectors resemble viral vectors but comprise two copies of the 3′ LTR generated during reverse transcription, see e.g., Pluta and Kacprzak, 2009.

In particular embodiments, a viral vector comprises a 5′ LTR, a packaging signal, a cPPT/FLAP element, a RNA export element, a transgene, and a 3′ LTR. Viral vectors may optionally comprise post-trascriptional regulatory elements and polyadenylation signals/sequences.

The term “long terminal repeat (LTR)” refers to domains of base pairs located at the ends of retroviral genomes which, in their natural sequence context, are direct repeats and contain U3, R and U5 regions. LTRs generally provide functions fundamental to the expression of retroviral genes (e.g., promotion, initiation and polyadenylation of gene transcripts) and to viral replication. The LTR contains numerous regulatory signals including transcriptional control elements, polyadenylation signals and sequences needed for replication and integration of the viral genome. The viral LTR is divided into three regions called U3, R and U5. The U3 region contains the enhancer and promoter elements. The U5 region is the sequence between the primer binding site and the R region and contains the polyadenylation sequence. The R (repeat) region is flanked by the U3 and U5 regions. The LTR composed of U3, R and U5 regions and appears at both the 5′ and 3′ ends of the viral genome. Adjacent to the 5′ LTR are sequences necessary for reverse transcription of the genome (the tRNA primer binding site) and for efficient packaging of viral RNA into particles (the Psi site).

As used herein, the term “modified LTR” refers to one or more nucleotide additions, deletions or substitutions in a native viral 5′ LTR and/or 3′ LTR. The skilled artisan would be able to determine whether an LTR is modified by comparison to a reference LTR. Either or both of the LTR may comprise one or more modifications. Modifications of the 3′ LTR are often made to improve the safety of viral vector systems, including but not limited to rendering viral vectors replication-defective.

As used herein, the term “replication-defective” refers to a viral vector that is not capable of complete, effective replication such that infective viral particles are not produced (e.g., replication-defective viral progeny).

“Self-inactivating” (SIN) viral vectors refers to replication-defective vectors, e.g., retroviral or lentiviral vectors, in which the right (3′) LTR enhancer-promoter region, known as the U3 region, has been modified (e.g., by deletion or substitution) to prevent viral transcription beyond the first round of viral replication. This is because the right (3′) LTR U3 region is used as a template for the left (5′) LTR U3 region during viral replication and, thus, the viral transcript cannot be made without the U3 enhancer-promoter. Self-inactivation is preferably achieved through in the introduction of a deletion in the U3 region of the 3′ LTR of the vector DNA, i.e., the DNA used to produce the vector RNA. Thus, during reverse transcription, this deletion is transferred to the 5′ LTR of the proviral DNA. In the case of lentiviral vectors, it has been discovered that such vectors tolerate significant U3 deletions, including the removal of the LTR TATA box (e.g., deletions from -418 to -18), without significant reductions in vector titers.

An additional safety enhancement, which also has been shown to increase viral titer, is provided by replacing the U3 region of the 5′ LTR with a heterologous promoter (i.e., chimeric 5′ LTR) to drive transcription of the viral vector genome during viral vector production. Chimeric 5′ LTR promoters are able to drive high levels of transcription in a Tat-independent manner. Illustrative examples of heterologous promoters suitable for use in particular embodiments contemplated herein include, but are not limited to simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters.

The “R region” refers to the region within LTRs beginning at the start of the capping group (i.e., the start of transcription) and ending immediately prior to the start of the poly A tract. The R region is also defined as being flanked by the U3 and U5 regions. The R region plays a role during reverse transcription in permitting the transfer of nascent DNA from one end of the genome to the other.

Viral vector contemplated in particular embodiments comprise a TAR element. The term “TAR” refers to the “trans-activation response” genetic element located in the R region of LTRs. This element interacts with the trans-activator (tat) genetic element to enhance viral replication. However, this element is not required in viral vectors wherein the U3 region of the 5′ LTR is replaced by a heterologous promoter.

As used herein, the term “packaging signal” or “packaging sequence” refers to sequences located within the viral genome which are required for insertion of the viral RNA into the viral capsid or particle, see e.g., Clever et al., 1995. J. of Virology, Vol. 69, No. 4; pp. 2101-2109. Several viral vectors use the minimal packaging signal (also referred to as the psi [Ψ] or [Ψ+] sequence) needed for encapsidation of the viral genome. Thus, as used herein, the terms “packaging sequence,” “packaging signal,” “psi” and the symbol “Ψ,” are used in reference to the non-coding sequence required for encapsidation of viral RNA strands during viral particle formation.

As used herein, the term “FLAP element” or “cPPT/FLAP” refers to a nucleic acid whose sequence includes the central polypurine tract and central termination sequences (cPPT and CTS) of a viral vector. Suitable FLAP elements are described in U.S. Pat. No. 6,682,907 and in Zennou, et al., 2000, Cell, 101:173. During viral reverse transcription, central initiation of the plus-strand DNA at the central polypurine tract (cPPT) and central termination at the central termination sequence (CTS) lead to the formation of a three-stranded DNA structure; the central DNA FLAP. While not wishing to be bound by any theory, the DNA FLAP may act as a cis-active determinant of viral vector genome nuclear import and/or may increase the titer of the virus.

The term “export element” refers to a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65: 1053; and Cullen et al., 1991. Cell 58: 423).

As used herein, the terms “post-transcriptional regulatory element” or “PRE” refer to a cis-acting element that regulates expression at the mRNA level by, for example, regulating capping, splicing, poly(A) tail addition, and mRNA stability. Illustrative examples of PTE include, but are not limited to, woodchuck hepatitis virus post-transcriptional regulatory element (WPRE; Zufferey et al., 1999, J. Virol., 73:2886); the post-transcriptional regulatory element present in hepatitis B virus (HPRE) (Huang and Yen, 1995, Mol. Cell. Biol., 5:3864); and the like (Liu et al., 1995, Genes Dev., 9:1766).

The term “poly(A) site” or “poly(A) sequence” as used herein denotes a DNA sequence which directs both the termination and polyadenylation of the nascent RNA transcript by RNA polymerase II. Polyadenylation sequences can promote mRNA stability by addition of a poly (A) tail to the 3′ end of the coding sequence and thus, contribute to increased translational efficiency. Cleavage and polyadenylation is directed by a poly(A) sequence in the RNA. The core poly (A) sequence for mammalian pre-mRNAs has two recognition elements flanking a cleavage-polyadenylation site. Typically, an almost invariant AAUAAA hexamer lies 20-50 nucleotides upstream of a more variable element rich in U or GU residues. Cleavage of the nascent transcript occurs between these two elements and is coupled to the addition of up to 250 adenosines to the 5′ cleavage product. In particular embodiments, the core poly(A) sequence is a synthetic poly(A) sequence (e.g., AATAAA, ATTAAA, AGTAAA).

As used herein, the terms “polynucleotide” or “nucleic acid” refer to deoxyribonucleic acid (DNA), ribonucleic acid (RNA) and DNA/RNA hybrids. Polynucleotides may be single-stranded or double-stranded and either recombinant, synthetic, or isolated. Polynucleotides include, but are not limited to: pre-messenger RNA (pre-mRNA), messenger RNA (mRNA), RNA, genomic RNA (gRNA), plus strand RNA (RNA(+)), minus strand RNA (RNA(−)), synthetic RNA, synthetic mRNA, genomic DNA (gDNA), PCR amplified DNA, complementary DNA (cDNA), synthetic DNA, or recombinant DNA. Illustrative examples of polynucleotides contemplated in particular embodiments include but are not limited to transfer plasmids, plasmids encoding viral structural and/or accessory proteins, e.g., gag, poly, tat, rev, and/or env, and polynucleotide(s)-of-interest.

As used herein, the terms “polynucleotide variant” and “variant” and the like refer to polynucleotides having at least about 50%, 55%, 60%, 65%, 70%, 75%, 80%, 85%, 90%, 95%, 96%, 97%, 98%, 99% or 100% sequence identity to any of the reference sequences described herein, typically where the variant maintains at least one biological activity of the reference sequence. Polynucleotide variants may include polynucleotides in which one or more nucleotides have been added or deleted, or replaced with different nucleotides compared to a reference polynucleotide. In this regard, it is well understood in the art that certain alterations inclusive of mutations, additions, deletions and substitutions can be made to a reference polynucleotide whereby the altered polynucleotide retains the biological function or activity of the reference polynucleotide.

As used herein, the term “polynucleotide(s)-of-interest” refers to one or more polynucleotides, e.g., a polynucleotide encoding a polypeptide (i.e., a polypeptide-of-interest), including a therapeutic polypeptide, inserted into a vector that is desired to be expressed. In particular embodiments, vectors and/or plasmids comprise one or more polynucleotides-of-interest that encode one or more therapeutic RNAs, e.g., shRNAs, miRNAs, or shmiRs, and/or therapeutic polypeptides.

Polynucleotides, regardless of the length of the coding sequence itself, may be combined with other DNA sequences, such as promoters and/or enhancers, untranslated regions (UTRs), Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, and polynucleotides encoding self-cleaving polypeptides, epitope tags, as disclosed elsewhere herein or as known in the art, such that their overall length may vary considerably. It is therefore contemplated that a polynucleotide fragment of almost any length may be employed, with the total length preferably being limited by the ease of preparation and use in the intended recombinant DNA protocol.

The term “operably linked”, refers to a juxtaposition wherein the components described are in a relationship permitting them to function in their intended manner.

The terms “polypeptide” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. Illustrative examples of polypeptides include, but are not limited to globin polypeptides, suitable for use in the compositions and methods of particular embodiments. Also, see, e.g., U.S. Pat. Nos. 6,051,402; 7,901,671; and 9,068,199, the full disclosure and claims of which are specifically incorporated herein by reference in their entireties.

Particular embodiments contemplated herein, also include polypeptide “variants.” The recitation polypeptide “variant” refers to polypeptides that are distinguished from a reference polypeptide by the addition, deletion, truncations, modifications, and/or substitution of at least one amino acid residue, and that retain a biological activity. In certain embodiments, a polypeptide variant is distinguished from a reference polypeptide by one or more substitutions, which may be conservative or non-conservative, as known in the art. In certain embodiments, a variant polypeptide includes an amino acid sequence having at least about 50%, 55%, 60%, 65%, 70%, 75%, 80%, 85%, 90%, 91%, 92%, 93%, 94%, 95%, 96%, 97%, 98%, 99% or more sequence identity or similarity to a corresponding sequence of a reference polypeptide. In certain embodiments, amino acid additions or deletions occur at the C-terminal end and/or the N-terminal end of the reference polypeptide.

Additional definitions are set forth throughout this disclosure.

### C. Upstream Viral Vector Manufacturing Process

The upstream manufacturing processes contemplated herein comprise culturing a population of host cells in a large working volume bioreactor, transfecting the host cells with a mixture comprising a transfer plasmid, packaging plasmids and a transfection agent; and culturing the transfected host cells to produce viral vector. In particular embodiments, the upstream manufacturing processes contemplated herein can be used in combination with various downstream manufacturing processes.

Upstream manufacturing processes contemplated in particular embodiments herein comprise thawing a working cell bank of host cells; culturing and expanding host cells in progressively larger volumes until a sufficient amount of host cells to seed a large working volume bioreactor; seeding the large working volume bioreactor with the host cells and culturing the host cells until they reach a sufficient density for transfection; exchanging the culture medium in the bioreactor; transfecting the host cells with a mixture comprising a transfection agent, packaging plasmids encoding viral accessory genes and a transfer plasmid encoding a packageable viral vector genome comprising a therapeutic transgene; culturing the transfected cells for a duration sufficient to complete transfection; exchanging the culture medium in the bioreactor; and culturing the cells for about one to about three days to produce the viral vector.

Upstream manufacturing processes contemplated in particular embodiments herein comprise thawing a working cell bank of host cells; culturing and expanding host cells in progressively larger volumes until a sufficient amount of host cells to seed a large working volume bioreactor; seeding the large working volume bioreactor with the host cells and culturing the host cells until they reach a sufficient density for transfection; exchanging the culture medium in the bioreactor; transfecting the host cells with a mixture comprising a transfection agent, packaging plasmids encoding viral accessory genes and a transfer plasmid encoding a packageable viral vector genome comprising a therapeutic transgene; culturing the transfected cells for a duration sufficient to complete transfection; exchanging the culture medium in the bioreactor; and culturing the cells for about one to about three days to produce the viral vector, e.g., retroviral or lentiviral vector.

In various embodiments, an upstream manufacturing process for a retroviral vector, e.g., lentiviral vector, comprises thawing a working cell bank of host cells, expanding the host cells in progressively larger volumes in serum free chemically defined cell culture medium over a period of about 18 days; inoculating a large working volume bioreactor, e.g., at least 200 liters working volume, with an amount of viable host cells and culturing the host cells in serum free chemically defined cell culture medium for about three days; exchanging the cell culture medium in the bioreactor with fresh serum free chemically defined cell culture medium; transfecting the cells with a mixture comprising a transfection agent, packaging plasmids encoding viral accessory genes and a transfer plasmid encoding a packageable viral vector genome comprising a therapeutic transgene; exchanging the culture medium in the bioreactor between about 12 hours to about 20 hours of transfection or between about 14 hours to about 18 hours of transfection with fresh serum free chemically defined cell culture medium; and culturing the transfected host cells for about one or about three days to produce the retroviral vector. In preferred embodiments, the culture supernatant is collected once during the about one or about three days retroviral production. In particular embodiments, the culture supernatant is collected one, two, or three times during the about one to about three days of retroviral vector production.

### 1. Host Cells

Large-scale viral vector production processes contemplated herein comprise introducing a transfer vector and plasmids encoding viral structural and/or accessory genes, e.g., gag, pol, env, rev, tat, vif, vpr, vpu, vpx, and/or nef genes, into a population of host cells.

A “host cell” refers to a cell that is modified to produce a viral vector. In particular embodiments, host cells include packaging cells and producer cells. A “packaging cell” is a host cell modified to express viral structural and/or accessory genes that enable packaging of a viral vector genome into a viral vector. A packaging cell does not contain a packaging signal to package the viral vector genome into a viral vector. A “producer cell” is a packaging cell that contains a viral vector genome comprising a packaging signal to package the viral vector genome into a viral vector.

In preferred embodiments, host cells are mammalian cells that can be cultured in suspension culture or that are capable of being adapted to suspension culture.

In particular embodiments, host cells suitable for use in particular embodiments, include but are not limited to CHO cells, A549 cells, and HEK293 cells and derivatives thereof. In preferred embodiments, the host cells are selected from the group consisting of: HEK293 cells, HEK293S cells, HEK293T cells adapted for suspension culture (HEK392Ts), HEK293F cells, HEK293FT cells, HEK293FTM cells, and HEK293E cells. In more preferred embodiments, the host cells are HEK293Ts cells.

### 2. Expansion Culture

Host cells are often stored in aliquots as part of a working cell bank. The working cell bank is a convenient method of storing substantially similar aliquots of host cells to maximize reproducibility in host cell expansion culture. The upstream manufacturing processes contemplated in particular embodiments, contemplated thawing host cells and culturing the host cells in progressively larger volumes to produce a sufficient number of viable host cells to seed a large-scale bioreactor. Large-scale bioreactors suitable for use in particular embodiments, include but are not limited to bioreactors with working volumes of at least 100 L, at least 200 L, at least 250 L, at least 500 L, at least 1000 L, at least 1500 L, or at least 2000 L.

In particular embodiments, a working cell bank is thawed and host cells are expanded (or passaged) through 1, 2, 3, 4, 5, 6 or more rounds of culture (e.g., P0, P1, P2, P3, P4, P5) in progressively larger volumes over a total time of about 10, 11, 12, 13, 14, 15, 16, 17, 18, 19, 20, 21, 22, 23, or 24 days.

Host cells are cultured for expansion in chemically defined cell culture media. In preferred embodiments, host cells are cultured in serum free chemically defined cell culture media. Illustrative examples of serum free chemically defined cell culture media suitable for use in particular embodiments include but are not limited to Freestyle 293 expression medium, Ex-Cell 293 serum free medium, Expi293 expression medium, and Opti-MEM reduced serum medium. Host cells are cultured at about 37° C. and at about pH 7.

In particular embodiments, a working cell bank is thawed and host cells are cultured in a volume of about 50 mL of medium (e.g., in a culture vessel of at least 250 mL) until the host cell culture reaches at least about 70%, at least about 75%, at least about 80%, at least about 85%, or at least about 90% viable cells at a viable cell density of at least about 0.5×10⁶ cells/mL, at least about 1×10⁶ cells/mL, at least about 1.5×10⁶ cells/mL, at least about 2×10⁶ cells/mL, or at least about 2.5×10⁶ cells/mL. In particular embodiments, the 50 mL culture is referred to as the P0 (first passage) culture.

In particular embodiments, after the viable host cells from the P0 culture reach a sufficient density, the cells are expanded to a culture volume of about 100 mL of medium (e.g., in a culture vessel of at least 500 mL) until the host cell culture reaches at least about 70%, at least about 75%, at least about 80%, at least about 85%, or at least about 90% viable cells a viable cell density of at least about 2.5×10⁶ cells/mL, at least about 3×10⁶ cells/mL, at least about 3.5×10⁶ cells/mL, at least about 4×10⁶ cells/mL, at least about 4.5×10⁶ cells/mL, at least about 5×10⁶ cells/mL, at least about 5.5×10⁶ cells/mL, at least about 6×10⁶ cells/mL, at least about 6.5×10⁶ cells/mL, at least about 7×10⁶ cells/mL, at least about 7.5×10⁶ cells/mL, at least about 8×10⁶ cells/mL, 8.5×10⁶ cells/mL, at least about 9×10⁶ cells/mL, at least about 9.5×10⁶ cells/mL, at least about 10×10⁶ cells/mL, at least about 10.5×10⁶ cells/mL, at least about 11×10⁶ cells/mL, at least about 115×10⁶ cells/mL, at least about 12×10⁶ cells/mL, at least about 12.5×10⁶ cells/mL, at least about 13×10⁶ cells/mL, at least about 13.5×10⁶ cells/mL, at least about 14×10⁶ cells/mL, at least about 14.5×10⁶ cells/mL, or at least about 15×10⁶ cells/mL. In particular embodiments, the 100 mL culture is referred to as the P1 (second passage) culture.

In particular embodiments, after the viable host cells from the P1 culture reach a sufficient density, the cells are expanded to one, two or three cultures, each with volume of about 200 mL of medium (e.g., in a culture vessel of at least 1 L) until the host cell cultures reach at least about 70%, at least about 75%, at least about 80%, at least about 85%, or at least about 90% viable cells at a viable cell density of at least about 2.5×10⁶ cells/mL, at least about 3×10⁶ cells/mL, at least about 3.5×10⁶ cells/mL, at least about 4×10⁶ cells/mL, at least about 4.5×10⁶ cells/mL, at least about 5×10⁶ cells/mL, at least about 5.5×10⁶ cells/mL, at least about 6×10⁶ cells/mL, at least about 6.5×10⁶ cells/mL, at least about 7×10⁶ cells/mL, at least about 7.5×10⁶ cells/mL, at least about 8×10⁶ cells/mL, 8.5×10⁶ cells/mL, at least about 9×10⁶ cells/mL, at least about 9.5×10⁶ cells/mL, at least about 10×10⁶ cells/mL, at least about 10.5×10⁶ cells/mL, at least about 11×10⁶ cells/mL, at least about 115×10⁶ cells/mL, at least about 12×10⁶ cells/mL, at least about 12.5×10⁶ cells/mL, at least about 13×10⁶ cells/mL, at least about 13.5×10⁶ cells/mL, at least about 14×10⁶ cells/mL, at least about 14.5×10⁶ cells/mL, or at least about 15×10⁶ cells/mL. In particular embodiments, the 200 mL cultures are referred to as the P2 (third passage) cultures.

In particular embodiments, after the viable host cells from the P2 culture(s) reach a sufficient density, the cells are expanded to one, two or three cultures, each with volume of about 1 L of medium (e.g., in a culture vessel of at least 3 L) until the host cell cultures reach at least about 70%, at least about 75%, at least about 80%, at least about 85%, or at least about 90% viable cells at a viable cell density of at least about 2.5×10⁶ cells/mL, at least about 3×10⁶ cells/mL, at least about 3.5×10⁶ cells/mL, at least about 4×10⁶ cells/mL, at least about 4.5×10⁶ cells/mL, at least about 5×10⁶ cells/mL, at least about 5.5×10⁶ cells/mL, at least about 6×10⁶ cells/mL, at least about 6.5×10⁶ cells/mL, at least about 7×10⁶ cells/mL, at least about 7.5×10⁶ cells/mL, at least about 8×10⁶ cells/mL, 8.5×10⁶ cells/mL, at least about 9×10⁶ cells/mL, at least about 9.5×10⁶ cells/mL, at least about 10×10⁶ cells/mL, at least about 10.5×10⁶ cells/mL, at least about 11×10⁶ cells/mL, at least about 115×10⁶ cells/mL, at least about 12×10⁶ cells/mL, at least about 12.5×10⁶ cells/mL, at least about 13×10⁶ cells/mL, at least about 13.5×10⁶ cells/mL, at least about 14×10⁶ cells/mL, at least about 14.5×10⁶ cells/mL, or at least about 15×10⁶ cells/mL. In particular embodiments, the 1 L cultures are referred to as the P3 (fourth passage) cultures.

In particular embodiments, after the viable host cells from the P3 culture(s) reach a sufficient density, the cells are expanded to culture in a bioreactor in a volume of about 20 L of medium (e.g., in a bioreactor with a volume of at least 50 L) until the host cell culture reaches at least about 85% or at least about 90% viable cells at a viable cell density of at least about 2.5×10⁶ cells/mL, at least about 3×10⁶ cells/mL, at least about 3.5×10⁶ cells/mL, at least about 4×10⁶ cells/mL, at least about 4.5×10⁶ cells/mL, at least about 5×10⁶ cells/mL, at least about 5.5×10⁶ cells/mL, at least about 6×10⁶ cells/mL, at least about 6.5×10⁶ cells/mL, at least about 7×10⁶ cells/mL, at least about 7.5×10⁶ cells/mL, at least about 8×10⁶ cells/mL, 8.5×10⁶ cells/mL, at least about 9×10⁶ cells/mL, at least about 9.5×10⁶ cells/mL, at least about 10×10⁶ cells/mL, at least about 10.5×10⁶ cells/mL, at least about 11×10⁶ cells/mL, at least about 115×10⁶ cells/mL, at least about 12×10⁶ cells/mL, at least about 12.5×10⁶ cells/mL, at least about 13×10⁶ cells/mL, at least about 13.5×10⁶ cells/mL, at least about 14×10⁶ cells/mL, at least about 14.5×10⁶ cells/mL, or at least about 15×10⁶ cells/mL. In particular embodiments, the 20 L culture is referred to as the P4 (fifth passage) culture.

In particular embodiments, after the viable host cells from the P4 culture(s) reach a sufficient density, a large-scale bioreactor (P5, sixth passage culture) with a working volume of at least about 200 L is seeded with at least about 0.1×10⁶ viable cells/mL, at least about 0.15×10⁶ viable cells/mL, at least about 0.2×10⁶ viable cells/mL, at least about 0.25×10⁶ viable cells/mL, at least about 0.3×10⁶ viable cells/mL, at least about 0.35×10⁶ viable cells/mL, at least about 0.4×10⁶ viable cells/mL, at least about 0.45×10⁶ viable cells/mL, or at least about 0.5×10⁶ viable cells/mL.

In various embodiments, large-scale suspension bioreactor is seeded/inoculated with about 40.0×10⁸ to about 120.0×10⁸ viable host cells. In some embodiments, large-scale suspension bioreactor is seeded/inoculated with about 50.0×10⁸ to about 120.0×10⁸ viable host cells. In some embodiments, large-scale suspension bioreactor is seeded/inoculated with about 60.0×10⁸ to about 120.0×10⁸ viable host cells. In some embodiments, large-scale suspension bioreactor is seeded/inoculated with about 70.0×10⁸ to about 120.0×10⁸ viable host cells. In some embodiments, large-scale suspension bioreactor is seeded/inoculated with about 80.0×10⁸ to about 120.0×10⁸ viable host cells. In some embodiments, large-scale suspension bioreactor is seeded/inoculated with about 90.0×10⁸ to about 120.0×10⁸ viable host cells. In some embodiments, large-scale suspension bioreactor is seeded/inoculated with about 100.0×10⁸ to about 120.0×10⁸ viable host cells. In some embodiments, large-scale suspension bioreactor is seeded/inoculated with about 110.0×10⁸ to about 120.0×10⁸ viable host cells. In some embodiments, large-scale suspension bioreactor is seeded/inoculated with about 40.0×10⁸ to about 110.0×10⁸ viable host cells. In some embodiments, large-scale suspension bioreactor is seeded/inoculated with about 40.0×10⁸ to about 100.0×10⁸ viable host cells. In some embodiments, large-scale suspension bioreactor is seeded/inoculated with about 40.0×10⁸ to about 90.0×10⁸ viable host cells. In some embodiments, large-scale suspension bioreactor is seeded/inoculated with about 40.0×10⁸ to about 80.0×10⁸ viable host cells. In some embodiments, large-scale suspension bioreactor is seeded/inoculated with about 40.0×10⁸ to about 70.0×10⁸ viable host cells. In some embodiments, large-scale suspension bioreactor is seeded/inoculated with about 40.0×10⁸ to about 60.0×10⁸ viable host cells. In some embodiments, large-scale suspension bioreactor is seeded/inoculated with about 40.0×10⁸ to about 50.0×10⁸ viable host cells. In some embodiments, large-scale suspension bioreactor is seeded/inoculated with about 50.0×10⁸ to about 110.0×10⁸ viable host cells. In some embodiments, large-scale suspension bioreactor is seeded/inoculated with about 60.0×10⁸ to about 100.0×10⁸ viable host cells. In some embodiments, large-scale suspension bioreactor is seeded/inoculated with about 70.0×10⁸ to about 90.0×10⁸ viable host cells. In some embodiments, large-scale suspension bioreactor is seeded/inoculated with about 40.0×10⁸ viable host cells. In some embodiments, large-scale suspension bioreactor is seeded/inoculated with about 50.0×10⁸ viable host cells. In some embodiments, large-scale suspension bioreactor is seeded/inoculated with about 60.0×10⁸ viable host cells. In some embodiments, large-scale suspension bioreactor is seeded/inoculated with about 70.0×10⁸ viable host cells. In some embodiments, large-scale suspension bioreactor is seeded/inoculated with about 80.0×10⁸ viable host cells. In some embodiments, large-scale suspension bioreactor is seeded/inoculated with about 90.0×10⁸ viable host cells. In some embodiments, large-scale suspension bioreactor is seeded/inoculated with about 100.0×10⁸ viable host cells. In some embodiments, large-scale suspension bioreactor is seeded/inoculated with about 110.0×10⁸ viable host cells. In some embodiments, large-scale suspension bioreactor is seeded/inoculated with about 120.0×10⁸ viable host cells.

The cells are cultured for about two, about three, or about four days, until the pre-transfection viable cell density is at least about 2.5×10⁶ cells/mL, at least about 3×10⁶ cells/mL, at least about 3.5×10⁶ cells/mL, at least about 4×10⁶ cells/mL, at least about 4.5×10⁶ cells/mL, at least about 5×10⁶ cells/mL, at least about 5.5×10⁶ cells/mL, at least about 6×10⁶ cells/mL, at least about 6.5×10⁶ cells/mL, at least about 7×10⁶ cells/mL, or at least about 7.5×10⁶ cells/mL.

In particular embodiments, the host cells cultured in the large-scale bioreactor reach the desired pre-transduction viable cell density and the cell culture medium in the bioreactor is exchanged for fresh chemically defined cell culture medium. In particular embodiments, an alternating tangential flow filtration (ATF) filter unit, a TFF filter unit, or an acoustic filter unit is used to perform the medium exchange. In one embodiment, an ATF filter unit is used to perform the medium exchange. In one embodiment, a TFF filter unit is used to perform the medium exchange. In one embodiment, an acoustic filter unit is used to perform the medium exchange. In preferred embodiments, after the bioreactor medium has been exchanged, the host cells are ready for transfection.

### 3. Transfection

Transfection is process of introducing one or more polynucleotides into a host cell by physical or chemical methods (nonviral). “Transfection” refers to the process of introducing naked DNA into cells by non-viral methods. Transfection can be stable or transient. In particular embodiments, host cells are transiently transfected with a mixture comprising one or more plasmids encoding one or more viral structural and/or accessory genes for packaging a viral vector genome, a transfer plasmid comprising packaging signal and a viral vector genome comprising a transgene (e.g., a therapeutic gene, gene of interest, or polynucleotide of interest), and a transfection agent.

A “transfection agent” is a molecule that increases the transfection of DNA into the host cell. Illustrative examples of transfection agents suitable for use in particular embodiments contemplated herein include but are not limited to calcium phosphate, cationic lipids, and cationic polymers.

Illustrative examples of cationic lipids suitable for use in particular embodiments contemplated herein include but are not limited to N-[1-(2,3-dioleoyloxy)propel]-N,N,N-trimethylammonium (DOTMA); 2,3-dioleyloxy-N-[2-spermine carboxamide] ethyl-N,N-dimethyl-1-propanammonium trifluoroacetate (DOSPA, Lipofectamine); 1,2-dioleoyl-3-trimethylammonium-propane (DOTAP); N-[1-(2,3-dimyristyloxy) propyl]-N,N-dimethyl-N-(2-hydroxyethyl) ammonium bromide (DMRIE), 3-β-[N-(N,N′-dimethylaminoethane) carbamoyl] cholesterol (DC-Chol); dioctadecyl amidoglyceryl spermine (DOGS, Transfectam); and imethyldioctadecylammonium bromide (DDAB). In particular embodiments, the cationic lipid is Lipofectamine.

Illustrative examples of cationic polymers suitable for use in particular embodiments contemplated herein include but are not limited to DEAE-dextran, poly brene, dendrimers, and polyethylenimine (PEI).

In particular embodiments, host cells in a large-scale bioreactor are transiently transfected with a mixture comprising a transfection agent, one or more plasmids encoding viral structural and/or accessory genes, and a transfer plasmid comprising a packageable viral vector genome encoding a human therapeutic transgene. In preferred embodiments, the transfection agent comprises a cationic polymer and in more preferred embodiments, the cationic polymer comprises PEI, and in even more preferred embodiments, the cationic polymer comprises a linear PEI.

In particular embodiments, the ratio of N (NH2 amines in PEI):P (phosphate groups in DNA backbone) for the PEI/DNA mixture is between about 5 and about 10, or about 5, about 5.5, about 6, about 6.4, about 6.5, about 7, about 7.5, about 8, about 8.5, about 9, about 9.5, or about 10. In some embodiments, the ratio of N (NH2 amines in PEI):P (phosphate groups in DNA backbone) for the PEI/DNA mixture is between about 5 and about 10. In some embodiments, the ratio of N (NH2 amines in PEI):P (phosphate groups in DNA backbone) for the PEI/DNA mixture is between about 5. In some embodiments, the ratio of N (NH2 amines in PEI):P (phosphate groups in DNA backbone) for the PEI/DNA mixture is between about 5.5. In some embodiments, the ratio of N (NH2 amines in PEI):P (phosphate groups in DNA backbone) for the PEI/DNA mixture is between about 6. In some embodiments, the ratio of N (NH2 amines in PEI):P (phosphate groups in DNA backbone) for the PEI/DNA mixture is between about 6.4. In some embodiments, the ratio of N (NH2 amines in PEI):P (phosphate groups in DNA backbone) for the PEI/DNA mixture is between about 6.5. In some embodiments, the ratio of N (NH2 amines in PEI):P (phosphate groups in DNA backbone) for the PEI/DNA mixture is between about 7. In some embodiments, the ratio of N (NH2 amines in PEI):P (phosphate groups in DNA backbone) for the PEI/DNA mixture is between about 7.5. In some embodiments, the ratio of N (NH2 amines in PEI):P (phosphate groups in DNA backbone) for the PEI/DNA mixture is between about 8. In some embodiments, the ratio of N (NH2 amines in PEI):P (phosphate groups in DNA backbone) for the PEI/DNA mixture is between about 9.5. In some embodiments, the ratio of N (NH2 amines in PEI):P (phosphate groups in DNA backbone) for the PEI/DNA mixture is between about 10.

In preferred embodiments, the one or more plasmids comprise polynucleotides encoding retroviral gag, pol, rev, a heterologous envelope protein, and optionally tat; more preferably, the one or more plasmids comprise polynucleotides encoding lentiviral gag, pol, rev, and a heterologous envelope protein.

In particular preferred embodiments, the one or more plasmids comprise a plasmid comprising a polynucleotide encoding lentiviral gag and pol, a plasmid encoding lentiviral rev, and a plasmid encoding a heterologous envelope glycoprotein including but not limited to an envelope glycoprotein from a Vesiculovirus envelope protein or variant thereof, Paramyxoviridae envelope protein or variant thereof, an Alphavirus envelope protein or variant thereof, a Gammaretrovirus envelope protein or variant thereof, an Orthohepadnavirus envelope protein or variant thereof, a Hepacivirus envelope protein or variant thereof, and a Lyssavirus envelope protein or variant thereof.

In particular preferred embodiments, the one or more plasmids comprise a plasmid comprising a polynucleotide encoding lentiviral gag and pol, a plasmid encoding lentiviral rev, and a plasmid encoding a heterologous envelope glycoprotein including but not limited to an envelope glycoprotein from a Vesicular stomatitis virus (VSV) envelope protein or variant thereof (e.g., VSV-G), a Cocal virus (COCV) envelope protein or variant thereof, a Maraba virus (MARAV) envelope protein or variant thereof, a Piry virus (PIRYV) envelope protein or variant thereof, a Nipah virus (NiV) envelope protein or variant thereof, a Sendai virus (SeV) envelope protein or variant thereof, a Morbillivirus envelope protein or variant thereof, a Canine distemper (CDV) envelope protein or variant thereof, a Measles virus (MV) envelope protein or variant thereof, a Sindbis virus (SINV) envelope protein or variant thereof, a Gibbon ape leukemia virus (GALV) envelope protein or variant thereof, a Feline endogenous retrovirus (RD114) envelope protein or variant thereof, a Feline leukemia virus (FeLV) envelope protein or variant thereof, a Baboon endogenous retrovirus (BaEV) envelope protein or variant thereof, a Hepatitis B (HBV) envelope protein or variant thereof, a Hepatitis C (HCV) envelope protein or variant thereof, and a Rabis virus (RABV) envelope protein or variant thereof.

In preferred embodiments, the transfer vector comprises an HIV lentiviral vector backbone comprising a packaging sequence and encoding a human therapeutic transgene; preferably, the transfer vector comprises an HIV-1 lentiviral vector backbone comprising a packaging sequence and encoding a human therapeutic transgene for treatment of a severe genetic disease or a cancer. In particular embodiments, the transfer vector comprises an HIV-1 lentiviral vector backbone comprising a packaging sequence and encoding a human therapeutic globin for treatment of a hemoglobinopathy, a ABCD1 gene for the treatment of CALD, or a chimeric receptor, e.g., a chimeric antigen receptor, a T cell receptor, or a DARIC for the treatment of a cancer.

In particular embodiments, host cells in a large-scale bioreactor are transiently transfected with a mixture comprising linear PEI, a plasmid encoding lentiviral gag and pol, a plasmid encoding rev, a plasmid encoding VSV-G and a transfer plasmid comprising a packageable HIV-1 based lentiviral vector genome encoding a human therapeutic transgene. In particular embodiments, the lentiviral gag and pol, rev, and/or VSV-G are codon optimized for expression and/or stability in human cells. In particular embodiments, the transfer plasmid and packaging plasmids comprise a selection cassette or gene. In preferred embodiments, the transfer plasmid and packaging plasmids comprise an RNAout selection cassette (Nature Technology). In particular embodiments, the host cells are transfected for about 12 hours, about 13 hours, about 14 hours, about 15 hours, about 16 hours, about 17 hours, about 18 hours, about 19 hours, or about 20 hours. In particular embodiments, the host cells are transfected for about 14 hours, about 15 hours, about 16 hours, about 17 hours, or about 18 hours. In more particular embodiments, the host cells are transfected for about 14 hours to about 18 hours.

After host cell transfection is complete, e.g., about 14 hours to about 18 hours post-transfection, an alternating tangential flow filtration (ATF) filter unit, a TFF filter unit, or an acoustic filter unit is used to perform the medium exchange. In one embodiment, an ATF filter unit is used to perform the medium exchange. In one embodiment, a TFF filter unit is used to perform the medium exchange. In one embodiment, an acoustic filter unit is used to perform the medium exchange. In particular embodiments, after the bioreactor medium has been exchanged, the host cells are cultured for viral vector production.

In particular embodiments, viral vector production occurs in serum free chemically defined cell culture medium from about 36 hours to about 48 hours post-transfection, about 36 hours to about 48 hours post-transfection, about 36 hours to about 46 hours post-transfection, about 36 hours to about 44 hours post-transfection, about 38 hours to about 48 hours post-transfection, about 38 hours to about 46 hours post-transfection, about 38 hours to about 44 hours post-transfection, or about 38 hours to about 42 hours post-transfection (e.g., 36 hours post-transfection is 36 hours after the addition of the transfection mixture to host cells). In particular embodiments, viral vector production occurs in serum free chemically defined cell culture medium until about 36 hours, about 37 hours, about 38 hours, about 39 hours, about 40 hours, about 41 hours, about 42 hours, about 43 hours, about 44 hours, about 45 hours, about 46 hours, about 47 hours or about 48 hours post-transfection. In particular embodiments, viral vector production occurs in serum free chemically defined cell culture medium until about 38 hours, about 39 hours, about 40 hours, about 41 hours, or about 42 hours post-transfection.

In particular embodiments, viral vector production occurs in serum free chemically defined cell culture medium for about 12 hours to about 48 hours, about 18 hours to about 48 hours, about 18 hours to about 36 hours, about 18 hours to about 30 hours, about 20 hours to about 28 hours, or about 22 hours to about 26 hours. In particular embodiments, viral vector production occurs in serum free chemically defined cell culture medium for about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours or about 48 hours.

In particular embodiments, the suspension culture supernatant is sampled and analyzed at one or more times to ensure sufficient viral vector production.

In various embodiments, the suspension culture supernatant is collected one, two, three or more times during the period of viral vector production. In particular embodiments, the suspension culture supernatant is collected once during the period of viral vector production. In preferred embodiments, after sufficient viral vector production, the suspension culture supernatant is not collected prior to initiation of the downstream manufacturing process.

### D. Downstream Viral Vector Manufacturing Process

Viral vector production marks the end of the upstream manufacturing process. The downstream manufacturing process begins at the end of the viral vector production period. The downstream manufacturing processes contemplated herein comprise adding a nuclease to the suspension culture supernatant at the end of the viral vector production period; harvesting and clarifying the suspension culture supernatant using filtration; capturing and concentrating the viral vector from the harvested and clarified suspension culture supernatant using chromatography; filtering the viral vector; ultrafiltering and diafiltering the viral vector; and formulating the viral vector. In particular embodiments, the bulk formulated viral vector is sterile filtered and frozen; subsequently thawed and sterile filtered; subjected to a final fill finish; and then frozen. In other particular embodiments, the bulk formulated viral vector is sterile filtered, subjected to a final fill finish; and then frozen

Downstream manufacturing processes contemplated in particular embodiments herein comprise adding a endonuclease to the suspension culture supernatant at the end of the viral vector production period; harvesting and clarifying the suspension culture supernatant using a tandem depth filtration; capturing and concentrating the viral vector from the harvested and clarified suspension culture supernatant using affinity chromatography or cation exchange chromatography; filtering the viral vector; ultrafiltering and diafiltering the viral vector using tangential flow filtration; and formulating the viral vector in cell culture medium. In particular embodiments, the bulk formulated viral vector is sterile filtered and frozen at ≤−65° C.; subsequently thawed and sterile filtered; subjected to a final fill finish; and then frozen at ≤−65° C. In other particular embodiments, the bulk formulated viral vector is sterile filtered, subjected to a final fill finish; and then frozen at ≤−65° C.

Downstream manufacturing processes contemplated in particular embodiments herein comprise adding a DNA endonuclease to the suspension culture supernatant at the end of the retroviral vector, e.g., lentiviral vector production period; harvesting and clarifying the suspension culture supernatant using tandem depth filtration and a second filter; capturing and concentrating the retroviral vector from the harvested and clarified suspension culture supernatant using heparin affinity chromatography or cation exchange chromatography; filtering the concentrated retroviral vector; ultrafiltering and diafiltering the retroviral vector using hollow fiber tangential flow filtration; and formulating the retroviral vector. In particular embodiments, the bulk formulated retroviral vector is sterile filtered and frozen at ≤−65° C.; subsequently thawed and sterile filtered; subjected to a final fill finish; and then frozen at ≤−65° C. In other particular embodiments, the bulk formulated retroviral vector is sterile filtered, subjected to a final fill finish; and then frozen at ≤−65° C.

In various embodiments, a downstream manufacturing process for retroviral vector production and purification comprises adding a DNA endonuclease, e.g., Benzonase or Denarase, to the suspension culture supernatant at the end of the retroviral vector, e.g., lentiviral vector production period; harvesting and clarifying the suspension culture supernatant using tandem depth filtration and a second filter, wherein the second filter comprises a prefilter membrane and a filtration membrane; capturing and concentrating the retroviral vector from the harvested and clarified suspension culture supernatant using heparin affinity chromatography or cation exchange chromatography; filtering the concentrated retroviral vector with a filter comprising a prefilter membrane and a filtration membrane; ultrafiltering and diafiltering the retroviral vector using hollow fiber tangential flow filtration, wherein the TFF has a molecular weight cutoff of about 100 kDa to about 500 kDa; and formulating the eluate containing the retroviral vector in formulation buffer (e.g., 2× Stem Cell Growth Medium (SCGM)), optionally, and in some embodiments preferably, in a 1:1 ratio. In particular embodiments, the bulk formulated retroviral vector is sterile filtered and frozen at ≤−65° C.; subsequently thawed and sterile filtered; subjected to a final fill finish; and then frozen at ≤−65° C. In other particular embodiments, the bulk formulated retroviral vector is sterile filtered, subjected to a final fill finish; and then frozen at ≤−65° C.

In particular embodiments, the downstream manufacturing processes contemplated herein result in at least about 10-fold, at least about 20-fold, at least about 30-fold, at least about 40-fold, at least about 50-fold, at least about 60-fold, at least about 70-fold, at least about 80-fold, at least about 90-fold, or at least about 100-fold or more viral vector concentration.

### 1. Nuclease Digestion

The upstream viral vector manufacturing process concludes after sufficient viral vector is produced. The viral vector supernatant may comprise residual nucleic acids including, but not limited to RNA, plasmid DNA from host cell transfection and genomic DNA from lysis of host cells during viral vector production. Such residual nucleic acids are potentially toxic and decrease the efficacy of any viral vector produced from the manufacturing processes contemplated herein. The purpose of the nuclease digestion step is to reduce the amount of these residual nucleic acids in the viral vector production supernatant.

In various embodiments, a nuclease is added to the viral vector production supernatant at the conclusion of the viral vector production process. In particular embodiments, the nuclease is an endonuclease, and in preferred embodiments, the nuclease is a DNA/RNA endonuclease (an endonuclease that cleaves both DNA and RNA). Illustrative examples of endonucleases suitable for use in particular embodiments of the downstream manufacturing processes contemplated herein include, but are not limited to Benzonase® endonuclease (EMD Millipore), Denarase® endonuclease (c-LEcta GmbH), Decontaminase™ endonuclease (AG Scientific), and recombinant NucA protein from Serratia marcescens.

In preferred embodiments, a Benzonase® endonuclease or recombinant NucA protein from Serratia marcescens is added to the viral vector production supernatant at the conclusion of the viral vector production process.

In particular embodiments, MgCl₂ is added with the endonuclease to the viral vector production supernatant to ensure that the endonuclease is catalytically active. In various embodiments, the endonuclease is added to the suspension culture supernatant about 36 hours to about 72 hours post-transfection. In some embodiments, the endonuclease is added to the suspension culture supernatant about 36 hours to about 48 hours post-transfection. In some embodiments, the endonuclease is added to the suspension culture supernatant about 48 hours post-transfection. In some embodiments, the endonuclease is added to the suspension culture supernatant about 44 hours post-transfection. In some embodiments, the endonuclease is added to the suspension culture supernatant about 40 hours after transfection. In some embodiments, the endonuclease is added to the suspension culture supernatant about 36 hours after transfection.

In particular embodiments, the nuclease digestion step of the downstream viral vector manufacturing process is performed at a suitable temperature and for a time sufficient to digest contaminating nucleic acids present in the viral vector production supernatant. In particular embodiments, nuclease digestion is performed from about 2° C. to about 8° C. overnight. In particular embodiments, nuclease digestion is performed from about 36° C. to about 38° C. for about one, about two, or about three hours. In preferred embodiments, endonuclease digestion is performed at about 36° C., about 37° C., or about 38° C. for about one, about two, or about three hours. In more preferred embodiments, Benzonase® endonuclease digestion is performed at about 37° C. for about 1 to 2 hours.

In various embodiments, the endonuclease digestion is performed at a concentration of about 20 U/ml to about 70 U/ml. In some embodiments, the endonuclease digestion is performed at a concentration of about 50 U/ml to about 70 U/ml. In some embodiments, the endonuclease digestion is performed at a concentration of about 55 U/ml to about 65 U/ml. In some embodiments, the endonuclease digestion is performed at a concentration of about 20 U/ml to about 40 U/ml. In some embodiments, the endonuclease digestion is performed at a concentration of about 25 U/ml to about 35 U/ml. In some embodiments, the endonuclease digestion is performed at a concentration of about 50 U/ml to about 70 U/ml. In some embodiments, the endonuclease digestion is performed at a concentration of about 20 U/ml. In some embodiments, the endonuclease digestion is performed at a concentration of about 25 U/ml. In some embodiments, the endonuclease digestion is performed at a concentration of about 30 U/ml. In some embodiments, the endonuclease digestion is performed at a concentration of about 35 U/ml. In some embodiments, the endonuclease digestion is performed at a concentration of about 40 U/ml. In some embodiments, the endonuclease digestion is performed at a concentration of about 45 U/ml. In some embodiments, the endonuclease digestion is performed at a concentration of about 50 U/ml. In some embodiments, the endonuclease digestion is performed at a concentration of about 55 U/ml. In some embodiments, the endonuclease digestion is performed at a concentration of about 60 U/ml. In some embodiments, the endonuclease digestion is performed at a concentration of about 65 U/ml. In some embodiments, the endonuclease digestion is performed at a concentration of about 70 U/ml.

After the nuclease has sufficiently digested the extracellular nucleic acids present in the viral vector production supernatant, the supernatant is clarified and filtered.

### 2. Clarification

Downstream viral vector manufacturing processes contemplated in particular embodiments further comprise a clarification step. The ultimate goal of clarification is to the preparation of the viral vector production supernatant for downstream chromatography and purification of the viral vector. The clarification step(s) remove contaminants, e.g., host cells and cell debris, prior to viral vector capture, chromatography, and purification.

In particular embodiments, the clarification step comprises a primary clarification step and a secondary clarification step. In particular embodiments, the primary clarification step comprises depth filtration and the secondary clarification step comprises membrane filtration.

A depth filter does not have a defined pore size or structure. Depth filters comprise gradient density structures specifically designed to retain particles of a defined size. The particles are retained within the whole depth of the filter media. Depth filter media may comprise cellulose, diatomaceous earth, or other materials suitable to retain contaminants of a particular size. Membrane filters, in contrast, retain particles of a particular size excluded by the pore size of the membrane at the membrane surface. In particular embodiments, membrane filters have a prefiltration membrane and a filtration membrane. In preferred embodiments, the prefiltration membranes have larger pore sizes than filtration membranes and function to reduce clogging or fouling of the filtration membrane. Multiple formats of depth filters and membrane filters are commercially available.

In various embodiments, harvesting and clarifying the suspension culture supernatant comprises a step of tandem depth filtration (primary clarification) and a membrane filtration step (secondary clarification).

In particular embodiments, a tandem depth filter retains contaminants of at least about 40 μm to about 60 μm. In particular embodiments, a tandem depth filter retains contaminants of at least about 40 μm, about 50 μm, or about 60 μm. In preferred embodiments, a tandem depth filter retains contaminants of at least about 60 μm. In preferred embodiments, a tandem depth filter retains contaminants of about 60 μm or more.

In particular embodiments, a tandem depth filter retains contaminants with a size greater than 40 μm to about 60 μm. In particular embodiments, a tandem depth filter retains contaminants with a size greater than about 40 μm, about 50 μm, or about 60 μm. In preferred embodiments, a tandem depth filter retains contaminants with a size greater than about 60 μm.

In particular embodiments, the membrane filtration is dual-layer filtration. In particular embodiments, the dual-layer filter comprising a prefilter membrane and a filtration membrane. In particular embodiments, the dual-layer filter comprises a prefilter membrane comprising a prefilter pore size of about 0.45 μm to about 0.8 μm and a final filtration membrane comprising a final filter pore size of about 0.22 μm to about 0.45 μm. In particular embodiments, the dual-layer filter comprises a prefilter membrane comprising a prefilter pore size of about 0.45 μm to about and a final filtration membrane comprising a final filter pore size of about 0.22 μm. In particular embodiments, the dual-layer filter comprises a prefilter membrane comprising a prefilter pore size of about 0.8 μm and a final filtration membrane comprising a final filter pore size of about 0.45 μm.

In various embodiments, harvesting and clarifying the suspension culture supernatant comprises a step of tandem depth filtration (primary clarification) using a tandem depth filter that retains contaminants with a size greater than about 40 μm, about 50 μm, or about 60 μm and a membrane filtration step (secondary clarification) using a dual-layer filter comprising a prefilter membrane with a prefilter pore size of about 0.45 μm to about 0.8 μm and a final filtration membrane with a final filter pore size of about 0.22 uμto about 0.45 μm.

In particular embodiments, harvesting and clarifying the suspension culture supernatant comprises a step of tandem depth filtration (primary clarification) using a tandem depth filter that retains contaminants with a size of about 60 μm or greater and a membrane filtration step (secondary clarification) using a dual-layer filter comprising a prefilter membrane with a prefilter pore size of about 0.45 μm and a final filtration membrane with a final filter pore size of to about 0.22 μm.

In particular embodiments, harvesting and clarifying the suspension culture supernatant comprises a step of tandem depth filtration (primary clarification) using a tandem depth filter that retains contaminants with a size of about 60 μm or greater and a membrane filtration step (secondary clarification) using a dual-layer filter comprising a prefilter membrane with a prefilter pore size of about 0.8 μm and a final filtration membrane with a final filter pore size of to about 0.45 μm. In various embodiments, the clarified suspension culture is adjusted to about pH 7.0 or pH 7.2 with 1M HEPES. In some embodiments, the clarified suspension culture is adjusted to about pH 7.0. In some embodiments, the clarified suspension culture is adjusted to about pH 7.2.

After the viral vector production supernatant has been harvested and clarified, it may optionally be stored at a suitable temperature, e.g., at about 4° C., about 20° C., about 30° C., or at about 37° C., for about 15, about 16, about 17, about 18, about 19, about 20, about 21, about 22, about 23, or about 24 hours. After harvesting and clarification, and optional storage, the viral vector is captured and concentrated using chromatography.

### 3. Chromatography

Downstream viral vector manufacturing processes contemplated in particular embodiments further comprise a chromatography step. Chromatography is usually performed on a column packed with a resin or bead designed to capture the viral vector from the harvested and clarified viral vector production supernatant and to allow the undesired impurities in the harvested and clarified viral vector production supernatant to pass through the column. Captured viral vector is then displaced or eluted from the column using desorption agents.

In particular embodiments, the chromatography is ion-exchange chromatography, size-exclusion chromatography, affinity chromatography, or multi-modal chromatography.

Ion exchange chromatography (IEX) involves the separation of ionizable molecules based on their total charge. IEX includes both anion exchange chromatography and cation exchange chromatography. Anion exchange chromatography (AEX) exploits the negatively charged surface of viral vector particles for purification purposes. AEX has been used to prepare inactivated HIV-1 vaccine and for the purification retroviral, e.g. lentiviral vectors. In particular embodiments, the viral vector from the harvested and clarified viral vector production supernatant is captured and concentrated using anion exchange chromatography. Cation exchange chromatography is another form of ion exchange chromatography (IEX). Cation exchange chromatography uses a negatively charged ion exchange resin with an affinity for molecules having net positive surface charges. Here, the pH of a lentiviral supernatant can be adjusted below the LVV iso-electric point to give the LVV an overall positive net surface charge which binds it to the negatively charged resin beads.

Accordingly, in various embodiments, the LVV supernatant is pumped over an ion-exchange chromatography column. In some embodiments, the LVV supernatant is pumped over a cation exchange chromatography column. In a particular embodiment, the LVV supernatant is pumped over a sulfate cation exchange chromatography (e.g., Toyopearl™ Sulfate-650F). In some embodiments, the sulfate cation exchange chromatography comprises a column having bead size of about 45 μm and/or a mean pore size of about 100 nm.

Size-exclusion chromatography (SEC) separates molecules based on their sizes using a resin that comprises beads with a defined pore size. Molecules elute from SEC resins in order of size; large molecules that are not trapped in bead pores travel a shorter distance and elute first and small molecules that are slowed by the bead pores elute last. Beads of different pore sizes can be purchased to achieve the desired resolution. SEC has been used to purify wild-type retroviruses and retroviral vectors. Retroviral vectors are excluded from the bead pores due to their large size and elute in the void volume of the column while lower molecular weight contaminants are retarded by the column and elute in later fractions. In particular embodiments, the viral vector from the harvested and clarified viral vector production supernatant is captured and concentrated using size-exclusion chromatography.

Affinity chromatography (AC) separates molecules based on their highly selective affinity for particular chromatographic adsorbents. Unfortunately, little is known about the composition of the viral membrane, which complicates the selection of suitable adsorbents. Viral vectors have been engineered to express affinity tags on their surface to facilitate purification, e.g., MoMLV modified to express hexahistidine affinity tags purified by immobilized metal affinity chromatography (IMAC). MoMLV viral vectors have also been purified by exploiting the interaction between streptavidin and biotin. Heparin affinity chromatography has been used to purify viral vectors that use heparan sulfate as cell surface receptor, including pseudotyped retroviral vectors, e.g., VSV-G pseudotyped lentiviral vectors. In particular embodiments, the viral vector from the harvested and clarified viral vector production supernatant is captured and concentrated using affinity chromatography.

Multimodal or mixed-mode chromatography (MMC) incorporates multiple modes of chromatography in a single resin. MMC enhances the selectivity of the resin because molecules can be separated based on several of their characteristics, rather than just a single one.

In preferred embodiments, the viral vector from the harvested and clarified viral vector production supernatant is captured and concentrated using heparin affinity chromatography. In particular embodiments, the harvested and clarified viral vector production supernatant is adjusted to a pH of about 7.0, if needed. The harvested and clarified viral vector production supernatant is passed over the heparin affinity chromatography column, the column is washed one or more times with wash buffer (e.g., 50 mM HEPES, 100 mM NaCl, pH 7.0) and eluted (e.g., 50 mM HEPES, 400 mM NaCl, pH 8.0).

In preferred embodiments, the viral vector from the harvested and clarified viral vector production supernatant is captured and concentrated using cation exchange chromatography. In various embodiments, the chromatography is a sulfate cation exchange chromatography. In particular embodiments, the harvested and clarified viral vector production supernatant is adjusted to a pH of about 7.2, if needed. The harvested and clarified viral vector production supernatant is passed/pumped over the sulfate cation exchange chromatography column, the column is washed one or more times with wash buffer (e.g., about 50 mM HEPES, about 300 mM NaCl, pH 7.2) and eluted (e.g., about 50 mM HEPES, about 1 M NaCl, pH 7.5).

The resulting eluate comprising the concentrated viral vector is filtered to further remove impurities.

### 4. Post-Chromatography Filtration

Downstream viral vector manufacturing processes contemplated in particular embodiments further comprise a filtration step following the chromatographic purification of the viral vector. The post-chromatography filtration protects unit operations downstream of the chromatography step by further removing impurities that may impede purification of the viral vector.

In particular embodiments, the filtering step comprises filtering the concentrated viral vector through a dual-layer filter comprising a pre-filter pore size of about 0.45 μm to about 0.8 μm and a final filter pore size of about 0.22 μm to about 0.45 μm.

In particular embodiments, the filtering step comprises filtering the concentrated viral vector through a dual-layer filter comprising a pre-filter pore size of about 0.45 μm and a final filter pore size of about 0.22 μm.

In particular embodiments, the filtering step comprises filtering the concentrated viral vector through a dual-layer filter comprising a pre-filter pore size of about 0.8 μm and a final filter pore size of about 0.45 μm.

In particular embodiments, after the concentrated viral vector is passed through the filter, the filter is chased with diafiltration buffer to maximize viral vector recovery.

The filtered viral vector solution is then further purified and concentrated using ultrafiltration and is then diafiltered.

### 5. Ultrafiltration/Diafiltration

Downstream viral vector manufacturing processes contemplated in particular embodiments further comprise an ultrafiltration step to further purify and concentrate the viral vector and a diafiltration step to exchange the buffer of the concentrated and filtered viral vector buffer to diafiltration buffer (e.g., about 50 mM HEPES, about 100 mM NaCl, pH 7.5; or about 50 mM HEPES, pH 7.0; or about 50 mM L-Histidine, pH 7.0) in preparation for formulation.

In particular embodiments, the viral vector is ultrafiltered to further remove impurities and concentrate the viral vector, and then subsequently diafiltered into a suitable buffer for bulk viral vector formulation.

In particular embodiments, viral vectors are filtered and concentrated and subsequently diafiltered using tangential flow filtration. In preferred embodiments, viral vectors are filtered and concentrated and subsequently diafiltered using tangential flow filtration. Hollow fiber TFF modules or filters have been used to simultaneously concentrate and remove impurities to yield highly active retroviral vectors. Hollow fiber TFF modules or filters have also been used as a convenient tool for diafiltering viral vectors into buffers suitable for bulk viral vector formulation.

In particular embodiments, the TFF systems comprise pumping the viral vector containing feed solution into the hollow fiber TFF module. The pore size of the TFF module is selected such that the viral vector does not pass through the pores and is concentrated in the retentate, the solution retained in the TFF module; whereas the permeate containing impurities passes through the pores.

In particular embodiments, the TFF systems are used to perform diafiltration or buffer exchange of a viral vector containing solution. TFF systems are an effective way to remove, modify, and/or exchange change ion concentration, pH, salts, sugars, non-aqueous solvents, separate unbound molecules, and remove low molecular weight contaminants.

In particular embodiments, a hollow fiber TFF module or filter is used to perform diafiltration and/or ultrafiltration to further purify, concentrate, and perform a buffer exchange. Suitable TFF systems for use in particular embodiments contemplated herein are commercially available, e.g., from EMD Millipore, Sigma, GE Healthcare, Sartorius, and Repligen.

In particular embodiments, hollow fiber TFF modules or filters comprise a pore size of about 100 kDa to about 500 kDa and a surface area of about 0.5 m², about 1.0 m², about 2.5 m², about 5.0 m², about 10 m², or about 20 m². In particular embodiments, hollow fiber TFF modules or filters comprise a pore size of about 100 kDa to about 500 kDa and a surface area of about 1.00 m², about 1.05 m², about 1.10 m², about 1.15 m², about 1.20 m², about 1.25 m², about 1.30 m², about 1.35 m², about 1.40 m², about 1.45 m², about 1.50 m², about 1.55 m², about 1.60 m², about 1.65 m², about 1.70 m², about 1.75 m², about 1.80 m², about 1.85 m², about 1.90 m², about 1.95 m², about 2.00 m², 2.00 m², about 2.05 m², about 2.10 m², about 2.15 m², about 2.20 m², about 2.25 m², about 2.30 m², about 2.35 m², about 2.40 m², about 2.45 m², or about 2.50 m².

In particular embodiments, hollow fiber TFF modules or filters comprise a pore size of about 100 kDa, about 200 kDa, about 300 kDa, about 400 kDa or to about 500 kDa and a surface area of about 1.00 m², about 1.05 m², about 1.10 m², about 1.15 m², about 1.20 m², about 1.25 m², about 1.30 m², about 1.35 m², about 1.40 m², about 1.45 m², about 1.50 m², about 1.55 m², about 1.60 m², about 1.65 m², about 1.70 m², about 1.75 m², about 1.80 m², about 1.85 m², about 1.90 m², about 1.95 m², about 2.00 m², 2.00 m², about 2.05 m², about 2.10 m², about 2.15 m², about 2.20 m², about 2.25 m², about 2.30 m², about 2.35 m², about 2.40 m², about 2.45 m², or about 2.50 m².

In particular embodiments, hollow fiber TFF modules or filters comprise a pore size of about 100 kDa, about 200 kDa, about 300 kDa, about 400 kDa or to about 500 kDa and a surface area of about 1.00 m², about 1.05 m², about 1.10 m², about 1.15 m², about 1.20 m², about 1.25 m², about 1.30 m², about 1.35 m², about 1.40 m², about 1.45 m², about 1.50 m², about 1.55 m², about 1.60 m², about 1.65 m², about 1.70 m², about 1.75 m², about 1.80 m², about 1.85 m², about 1.90 m², about 1.95 m², about 2.00 m², 2.00 m², about 2.05 m², about 2.10 m², about 2.15 m², about 2.20 m², about 2.25 m², about 2.30 m², about 2.35 m², about 2.40 m², about 2.45 m², or about 2.50 m².

In particular embodiments, hollow fiber TFF modules or filters comprise a pore size of about 100 kDa and a surface area of about 1.25 m², about 1.30 m², about 1.35 m², about 1.40 m², about 1.45 m², or about 1.50 m². In particular embodiments, hollow fiber TFF modules or filters comprise a pore size of about 200 kDa and a surface area of about 1.25 m², about 1.30 m², about 1.35 m², about 1.40 m², about 1.45 m², or about 1.50 m². In particular embodiments, hollow fiber TFF modules or filters comprise a pore size of about 300 kDa and a surface area of about 1.25 m², about 1.30 m², about 1.35 m², about 1.40 m², about 1.45 m², or about 1.50 m². In particular embodiments, hollow fiber TFF modules or filters comprise a pore size of about 400 kDa and a surface area of about 1.25 m², about 1.30 m², about 1.35 m², about 1.40 m², about 1.45 m², or about 1.50 m². In particular embodiments, hollow fiber TFF modules or filters comprise a pore size of about 500 kDa and a surface area of about 1.25 m², about 1.30 m², about 1.35 m², about 1.40 m², about 1.45 m², or about 1.50 m².

In particular embodiments, downstream viral vector manufacturing processes comprise an ultrafiltration step performed using a hollow fiber TFF module with a pore size of about 100 kDa, about 200 kDa, about 300 kDa, about 400 kDa, or about 500 kDa, and further comprise a diafiltration step performed using the hollow fiber TFF module to exchange the buffer containing the viral vector to a diafiltration buffer (e.g., 50 mM HEPES, 100 mM NaCl, pH 7.5; or 50 mM HEPES, pH 7.0) in preparation for formulation.

### 6. Formulation

Downstream viral vector manufacturing processes contemplated in particular embodiments further comprise formulating the viral vector in a suitable buffer and/or pharmaceutically acceptable medium. The viral vector is formulated to stabilize the vector and to retain vector activity through freeze/thaw cycles.

Ultrafiltration and diafiltration steps contemplated in particular embodiments result in purification, concentration and diafiltration of the viral vector into a diafiltration buffer (e.g., about 50 mM HEPES, about 100 mM NaCl, pH 7.5; or about 50 mM HEPES, pH 7.0; or about 50 mM L-Histidine, pH 7.0).

In particular embodiments, to formulate a viral vector the volume of diafiltrated viral vector is diluted in an equal volume of 2× concentrated a suitable formulation buffer or pharmaceutically cell culture medium. In particular embodiments, a viral vector is formulated by diluting an equal volume of diafiltered viral vector into 2× concentrated serum free chemically defined cell culture media. Illustrative examples of suitable formulation media include, but are not limited to 2× Freestyle 293 expression medium, 2× Ex-Cell 293 serum free medium, 2× Expi293 expression medium, 2× Opti-MEM reduced serum medium, and 2× Stem Cell Growth Medium (SCGM, CellGenix).

In certain embodiments, a viral vector is formulated by diluting an equal volume of diafiltered viral vector into 2× concentrated SCGM. In some embodiments, a viral vector diafiltered in 50 mM HEPES, 100 mM NaCl, pH 7.50 is formulated by diluting it 1:1 in 2× SCGM. In some embodiments, a viral vector diafiltered in about 50 mM HEPES, pH 7.0 is formulated by diluting 1:1 in a buffer comprising HEPES and Sucrose, optionally wherein the buffer further comprises L-proline, poloxamer 188, or NaCl. In some embodiments, a viral vector diafiltered in about 50 mM HEPES, pH 7.0 is formulated by diluting 1:1 in a buffer comprising about 5 mM HEPES (pH 7.0), about 146 mM Sucrose, and about 100 mM L-proline. In some embodiments, a viral vector diafiltered in about 50 mM HEPES, pH 7.0 is formulated by diluting 1:1 in a buffer comprising about 5 mM HEPES (pH 7.0), about 146 mM Sucrose, about 100 mM L-proline, and about 0.2 to about 2.0 mg/mL poloxamer 188. In some embodiments, a viral vector diafiltered in about 50 mM HEPES, pH 7.0 is formulated by diluting 1:1 in a buffer comprising about 5 mM HEPES (pH 7.0), about 146 mM Sucrose, about 100 mM L-proline, and about 150 mM NaCl. In some embodiments, a viral vector diafiltered in about 50 mM HEPES, pH 7.0 is formulated by diluting 1:1 in a buffer comprising about 5 mM HEPES (pH 7.0), about 146 mM Sucrose, about 100 mM L-proline, about 150 mM NaCl, and about 0.2 to about 2.0 mg/mL poloxamer 188. In some embodiments, a viral vector diafiltered in about 50 mM HEPES, pH 7.0 is formulated by diluting 1:1 in a buffer comprising about 5 mM HEPES (pH 7.0), about 146 mM Sucrose, about 150 mM NaCl, and about 0.2 to about 2.0 mg/mL poloxamer 188.

In various embodiments, a viral vector diafiltered in about 50 mM L-Histidine, pH 7.0 is formulated by diluting 1:1 in a buffer comprising L-Histidine, Sucrose, and L-proline. In various embodiments, a viral vector diafiltered in about 50 mM L-Histidine, pH 7.0 is formulated by diluting 1:1 in a buffer comprising about 5 mM L-Histidine, about 146 mM Sucrose, and about 100 mM L-proline, optionally wherein the formulation further comprises about 0.2 to about 2.0 mg/mL poloxamer 188.

In various embodiments, the formulation buffer for 1:1 dilution comprises about 0.2 mg/mL poloxamer 188. In some embodiments, the formulation buffer for 1:1 dilution comprises about 0.3 mg/mL poloxamer 188. In some embodiments, the formulation buffer for 1:1 dilution comprises about 0.4 mg/mL poloxamer 188. In some embodiments, the formulation buffer for 1:1 dilution comprises about 0.5 mg/mL poloxamer 188. In some embodiments, the formulation buffer for 1:1 dilution comprises about 0.6 mg/ml poloxamer 188. In some embodiments, the formulation buffer for 1:1 dilution comprises about 0.7 mg/mL poloxamer 188. In some embodiments, the formulation buffer for 1:1 dilution comprises about 0.8 mg/mL poloxamer 188. In some embodiments, the formulation buffer for 1:1 dilution comprises about 0.9 mg/mL poloxamer 188. In some embodiments, the formulation buffer for 1:1 dilution comprises about 1.0 mg/mL poloxamer 188. In some embodiments, the formulation buffer for 1:1 dilution comprises about 1.1 mg/mL poloxamer 188. In some embodiments, the formulation buffer for 1:1 dilution comprises about 1.2 mg/mL poloxamer 188. In some embodiments, the formulation buffer for 1:1 dilution comprises about 1.3 mg/mL poloxamer 188. In some embodiments, the formulation buffer for 1:1 dilution comprises about 1.4 mg/ml poloxamer 188. In some embodiments, the formulation buffer for 1:1 dilution comprises about 1.5 mg/mL poloxamer 188. In some embodiments, the formulation buffer for 1:1 dilution comprises about 1.6 mg/mL poloxamer 188. In some embodiments, the formulation buffer for 1:1 dilution comprises about 1.7 mg/mL poloxamer 188. In some embodiments, the formulation buffer for 1:1 dilution comprises about 1.8 mg/ml poloxamer 188. In some embodiments, the formulation buffer for 1:1 dilution comprises about 1.9 mg/mL poloxamer 188. In some embodiments, the formulation buffer for 1:1 dilution comprises about 2.0 mg/mL poloxamer 188.

The viral vector is formulated in bulk and is often, and in particular embodiments, preferably referred to as a formulated bulk viral vector.

### 7. Post-Formulation Filtration

Downstream viral vector manufacturing processes contemplated in particular embodiments further comprise a sterile filtration step following the formulation of the viral vector. The post-formulation filtration further removes particulates and impurities in the formulated bulk viral vector.

In particular embodiments, the filtering step comprises filtering the formulated bulk viral vector through a dual-layer filter comprising a pre-filter pore size of about 0.5 μm and a final filter pore size of about 0.2 μm.

In particular embodiments, after the formulated bulk viral vector is passed through the filter and recovered, the filtered formulated bulk viral vector is cryopreserved or subject to a hold step (without cryopreservation) until a final fill finish can be performed.

In particular embodiments, after the formulated bulk viral vector is passed through the filter and recovered, a final fill finish is performed on the filtered formulated bulk viral vector and subsequently cryopreserved.

### 8. Cryopreservation

Downstream viral vector manufacturing processes contemplated in particular embodiments further comprise cryopreserving a filtered formulated bulk viral vector until such a time as a final fill finish can be performed on the bulk viral vector. Cryopreservation of the formulated bulk viral vector is performed such that stability and biological activity of the vector is substantially maintained, and/or such that loss of viral vector stability and biological activity is minimized.

As used herein, “cryopreserving” or “cryopreservation” refers to the preservation of viral vector by cooling to sub-zero temperatures. In particular embodiments, a filtered formulated bulk viral vector further comprises a cryoprotective agent. In particular embodiments, the viral vector is formulated to provide cryoprotection.

In various embodiments, the filtered formulated bulk viral vector is cryopreserved or frozen at a temperature less than about −20° C., less than about −21° C., less than about −22° C., less than about −23° C., less than about −24° C., less than about −25° C., less than about −26° C., less than about −27° C., less than about −28° C., less than about −29° C., less than about −30° C., less than about −31° C., less than about −32° C., less than about −33° C., less than about −34° C., less than about −35° C., less than about −36° C., less than about −37° C., less than about −38° C., less than about −39° C., less than about −40° C., less than about −41° C., less than about −42° C., less than about −43° C., less than about −44° C., less than about −45° C., less than about −46° C., less than about −47° C., less than about −48° C., less than about −49° C., less than about −50° C., less than about −51° C., less than about −52° C., less than about −53° C., less than about −54° C., less than about −55° C., less than about −56° C., less than about −57° C., less than about −58°C., less than about −59° C., less than about −60° C., less than about −61° C., less than about −62° C., less than about −63° C., less than about −64° C., less than about −65° C., less than about −66° C., less than about −67° C., less than about −68° C., less than about −69° C., less than about −70° C., less than about −71° C., less than about −72° C., less than about −73° C., less than about −74° C., less than about −75° C., less than about −76° C., less than about −77° C., less than about −78° C., less than about −79° C., or less than about −80° C. The skilled understands that a temperature of −80° C. is less than a temperature of −20° C.

In various embodiments, the filtered formulated bulk viral vector is cryopreserved or frozen at a temperature less than about −65° C., less than about −70° C., less than about −75° C., or less than about −80° C.

In particular embodiments, the cooling rate is 1° to 3° C./minute.

### 9. Fill Finish

Downstream manufacturing processes contemplated herein further comprise a fill finish step. Viral vectors are typically aliquoted into single use volumes and cryopreserved to protect the stability and biological activity of the vector and minimize viral vector thermal inactivation.

A “fill finish” or “fill and finish” refers to part of a downstream manufacturing process that comprises filling containers, e.g., vials, ampules, etc., with formulated viral vector and finishing the process of packaging the viral vector for distribution.

In particular embodiments, a fill finish is performed on the filtered formulated viral vector without an intervening cryopreservation step. After the fill finish, the viral vector is cryopreserved according to the methods contemplated herein. In particular embodiments, the viral vector is cryopreserved or frozen at a temperature less than about −65° C., less than about −70° C., less than about −75° C., or less than about −80° C.

In particular embodiments, a fill finish is performed on the filtered formulated viral vector with an intervening hold step but not with an intervening cryopreservation step. After the fill finish, the viral vector is cryopreserved according to the methods contemplated herein. In particular embodiments, the viral vector is cryopreserved or frozen at a temperature less than about −65° C., less than about −70° C., less than about −75° C., or less than about −80° C.

In particular embodiments, a cryopreserved filtered formulated bulk viral vector is thawed and sterile filtered prior to a fill finish. In particular embodiments, the filtering step comprises filtering the thawed formulated bulk viral vector through a dual-layer filter comprising a pre-filter pore size of about 0.5 μm and a final filter pore size of about 0.2 μm. After filtering, a fill finish is performed with the formulated viral vector. After the fill finish, the viral vector is cryopreserved according to the methods contemplated herein. In particular embodiments, the viral vector is cryopreserved or frozen at a temperature less than about −65° C., less than about −70° C., less than about −75° ° C., or less than about −80° C.

### E. Compositions

The compositions contemplated herein may comprise a viral vector, e.g., a retroviral or lentiviral vector. Compositions include, but are not limited to pharmaceutical compositions. A “pharmaceutical composition” refers to a composition formulated in pharmaceutically-acceptable or physiologically-acceptable solutions for administration to a cell or an animal, either alone, or in combination with one or more other modalities of therapy. There is virtually no limit to other components that may also be included in the compositions, provided that the additional agents do not adversely affect the ability of the composition to deliver the intended therapy.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

In particular embodiments, compositions comprise a viral vector and a physiologically acceptable buffer or medium.

In particular embodiments, compositions comprise a viral vector and a diafiltration buffer (e.g., about 50 mM HEPES, about 100 mM NaCl, pH 7.5; or about 50 mM HEPES, pH 7.0; or about 50 mM L-Histidine, pH 7.0).

In particular embodiments, compositions comprise a viral vector and a pharmaceutically cell culture medium. Illustrative examples of suitable media include, but are not limited to 2× Freestyle 293 expression medium, 2× Ex-Cell 293 serum free medium, 2× Expi293 expression medium, 2× Opti-MEM reduced serum medium, and 2× Stem Cell Growth Medium (SCGM, CellGenix).

In certain embodiments, compositions comprise a viral vector, a diafiltration buffer and 1×SCGM.

In certain embodiments, composition comprise a viral vector in a HEPES-based formulation.

In certain embodiments, composition comprise a viral vector in a L-Histidine-based formulation.

In some embodiments, the composition comprises a viral vector, HEPES, and Sucrose, optionally wherein the buffer further comprises L-proline, poloxamer 188, or NaCl.

In some embodiments, the composition comprises a viral vector, about 27.5 mM HEPES (pH 7.0), about 73 mM Sucrose, and about 50 mM L-proline.

In some embodiments, the composition comprises a viral vector, about 27.5 mM HEPES (pH 7.0), about 73 mM Sucrose, about 50 mM L-proline, and about 0.1 to about 1.0 mg/ml poloxamer 188.

In some embodiments, the composition comprises a viral vector, about 27.5 mM HEPES (pH 7.0), about 73 mM Sucrose, about 50 mM L-proline, and about 75 mM NaCl.

In some embodiments, the composition comprises a viral vector, about 27.5 mM HEPES (pH 7.0), about 73 mM Sucrose, about 50 mM L-proline, about 75 mM NaCl, and about 0.1 to about 1.0 mg/ml poloxamer 188.

In some embodiments, the composition comprises a viral vector, about 27.5 mM HEPES (pH 7.0), about 73 mM Sucrose, about 75 mM NaCl, and about 0.1 to about 1.0 mg/ml poloxamer 188.

In some embodiments, the composition comprises a viral vector, L-Histidine, Sucrose, and L-proline.

In some embodiments, the composition comprises a viral vector, about 27.5 mM L-Histidine, about 73 mM Sucrose, and about 50 mM L-proline, optionally wherein the formulation further comprises about 0.1 to about 1.0 mg/mL poloxamer 188.

In some embodiments, the composition comprises about 0.1 mg/mL poloxamer 188. In some embodiments, the composition comprises about 0.2 mg/mL poloxamer 188. In some embodiments, the composition comprises about 0.3 mg/mL poloxamer 188. In some embodiments, the composition comprises about 0.4 mg/mL poloxamer 188. In some embodiments, the composition comprises about 0.5 mg/mL poloxamer 188. In some embodiments, the composition comprises about 0.6 mg/mL poloxamer 188. In some embodiments, the composition comprises about 0.7 mg/mL poloxamer 188. In some embodiments, the composition comprises about 0.8 mg/mL poloxamer 188. In some embodiments, the composition comprises about 0.9 mg/mL poloxamer 188. In some embodiments, the composition comprises about 1.0 mg/mL poloxamer 188.

It would be understood by the skilled artisan that particular embodiments of compositions contemplated herein may comprise other components including but not limited to those that are well known in the pharmaceutical art, and are described, for example, in Remington: The Science and Practice of Pharmacy, volume I and volume II. 22^{nd} Edition. Edited by Loyd V. Allen Jr. Philadelphia, PA: Pharmaceutical Press; 2012, which is incorporated by reference herein, in its entirety.

All publications, patent applications, and issued patents cited in this specification are herein incorporated by reference as if each individual publication, patent application, or issued patent were specifically and individually indicated to be incorporated by reference.

Although the foregoing embodiments have been described in some detail by way of illustration and example for purposes of clarity of understanding, it will be readily apparent to one of ordinary skill in the art in light of the teachings contemplated herein that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### Lentiviral Vector Suspension Manufacturing Process 2.0

A HEK293Ts working cell bank was cultured in a 250 mL culture vessel and cultured in a serum free chemically defined cell culture medium at 37.0° C. and 8.0% CO2. After a suitable viable cell density of this P0 culture was confirmed, the culture was passaged to a 500 mL culture vessel and cultured in a serum free chemically defined cell culture medium at 37.0° C. and 8.0% CO2. After a suitable viable cell density of this P1 culture was confirmed, the culture was passaged to a 1 L culture vessel and cultured in a serum free chemically defined cell culture medium at 37.0° C. and 8.0% CO2. After a suitable viable cell density of this P2 culture was confirmed, the culture was passaged to three 3 L culture vessels and cultured in a serum free chemically defined cell culture medium at 37.0° C. and 8.0% CO2. After a suitable viable cell density of this P3 culture was confirmed, the culture was passaged to a 50 L culture vessel and cultured in a serum free chemically defined cell culture medium at 37.0° C. and 8.0% CO2. After a suitable viable cell density of this P4 culture was confirmed, the culture was passaged to a 200 L bioreactor (P5 culture) and cultured in a serum free chemically defined cell culture medium at 37.0° C. and at pH 7.0. After a suitable viable cell density of this P5 culture was confirmed, the culture medium was exchanged with 190 L of fresh serum free chemically defined cell culture medium using alternating tangential flow filtration (ATF).

Prior to transfection, a transfer plasmid comprising a packageable lentiviral vector genome and gag/pol, rev, and VSV-G packaging plasmids and PEI components were mixed together in serum free chemically defined cell culture medium in a volume of 10 L. The DNA/PEI mixture was added to the P5 suspension culture for about 14 to about 18 hours, and subsequently subjected to a culture medium exchange with fresh serum free chemically defined cell culture medium using ATF.

About 36 to 48 hours post-transfection (after initiation of transfection), Benzonase endonuclease (final concentration of 50 to 75 U/mL) and MgCl₂ diluted in serum free chemically defined cell culture medium was added to the 200 L bioreactor culture and incubated for about 1 to 2 hours at 37° C.

The Benonzase treated culture was pumped through a tandem depth filter that retains contaminants greater than about 60 μm, then through a dual-layer filter that has a prefilter pore size of about 0.8 μm and a final filter of about 0.45 μm.

The clarified vector production supernatant was captured and concentrated using pseudo-affinity heparin chromatography. The supernatant was pumped over the chromatography column, washed, eluted in elution buffer (e.g., 50 mM HEPES, 400 mM NaCl, pH 8). The concentrated vector solution was pumped through a dual-layer filter that has a prefilter pore size of about 0.8 μm and a final filter of about 0.45 μm.

The filtered concentrated vector was pumped through a hollow fiber TFF column with a molecular weight cutoff or pore size of about 300 kDa to about 500 kDa. The retentate was then diafiltered against diafiltration buffer (e.g., 50 mM HEPES, 100 mM NaCl, pH 7.5) and formulated with a 1:1 dilution of 2×/1×SCGM formulation stock solution to form the formulated bulk lentiviral vector (LVV). The formulated bulk LVV was filtered with a sterilizing grade filter and filled into bulk storage containers and stored at ≤−65 ° C. until fill/finish was performed.

The frozen formulated bulk LVV was thawed and aliquots were pooled together and mixed before final filtration was performed followed by fill/finish using ready-to-use container/closure from West Pharmaceuticals and an automated filling line.

### Example 2

### Lentiviral Vector Suspension Manufacturing Process 2.5

A HEK293Ts working cell bank was cultured in a 250 mL culture vessel and cultured in a serum free chemically defined cell culture medium at 37.0° C. and 8.0% CO2. After a suitable viable cell density of this P0 culture was confirmed, the culture was passaged to a 500 mL culture vessel and cultured in a serum free chemically defined cell culture medium at 37.0° C. and 8.0% CO2. After a suitable viable cell density of this P1 culture was confirmed, the culture was passaged to a 1 L culture vessel and cultured in a serum free chemically defined cell culture medium at 37.0° C. and 8.0% CO2. After a suitable viable cell density of this P2 culture was confirmed, the culture was passaged to three 3 L culture vessels and cultured in a serum free chemically defined cell culture medium at 37.0° C. and 8.0% CO2. After a suitable viable cell density of this P3 culture was confirmed, the culture was passaged to a 50 L culture vessel and cultured in a serum free chemically defined cell culture medium at 37.0° C. and 8.0% CO2. After a suitable viable cell density of this P4 culture was confirmed, the culture was passaged to a 200 L bioreactor (P5 culture) and cultured in a serum free chemically defined cell culture medium at 37.0° C. and at pH 7.0. After a suitable viable cell density of this P5 culture was confirmed, the culture medium was exchanged with 190 L of fresh serum free chemically defined cell culture medium using alternating tangential flow filtration (ATF).

Prior to transfection, a transfer plasmid comprising a packageable lentiviral vector genome and gag/pol, rev, and VSV-G packaging plasmids and PEI components were mixed together in serum free chemically defined cell culture medium in a volume of 10 L. The DNA/PEI mixture was added to the P5 suspension culture for about 14 to about 18 hours, and subsequently subjected to a culture medium exchange with fresh serum free chemically defined cell culture medium using ATF.

About 36 to 48 hours post-transfection (after initiation of transfection), Denarase endonuclease (˜30 U/ml) and MgCl₂ diluted in serum free chemically defined cell culture medium was added to the 200 L bioreactor culture for about 1 to 2 hours at 37° C.

The Denarase treated culture was pumped through a tandem depth filter that retains contaminants greater than about 60 μm, then through a dual-layer filter that has a prefilter pore size of about 0.8 μm and a final filter of about 0.45 μm.

The clarified vector production supernatant was captured and concentrated using cation-exchange sulfate chromatography. The supernatant was pumped over the chromatography column, washed in wash buffer (e.g., 50 mM HEPES, 300 mM NaCl, pH 7.2), eluted in elution buffer (e.g., 50 mM HEPES, 1 M NaCl, pH 7.5). The concentrated vector solution was pumped through a dual-layer filter that has a prefilter pore size of about 0.8 μm and a final filter of about 0.45 μm.

The filtered concentrated vector was pumped through a hollow fiber TFF column with a molecular weight cutoff or pore size of about 300 kDa to about 500 kDa. The retentate was then diafiltered against diafiltration buffer (e.g., 50 mM HEPES, pH 7.0) and formulated with a 1:1 dilution of a concentrated formulation medium (e.g., 5 mM HEPES, 146 mM Sucrose, 100 mM L-proline, pH 7.0) to form the formulated bulk lentiviral vector (LVV). The formulated bulk LVV was filtered with a sterilizing grade filter and stored at 2 to 8° C. until fill/finish was performed.

The refrigerated intermediate bulk was sterile filtered, and fill/finish was performed using ready-to-use container/closure (West Pharmaceuticals) and an automated filling line.

### Example 3

### Lentiviral Vector Manufacturing Process Comparison

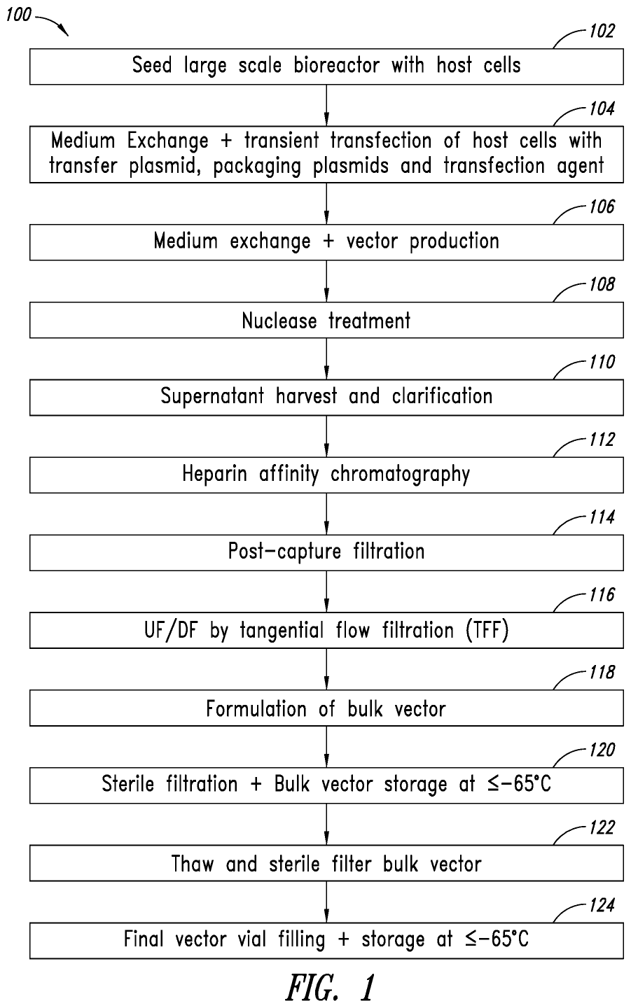

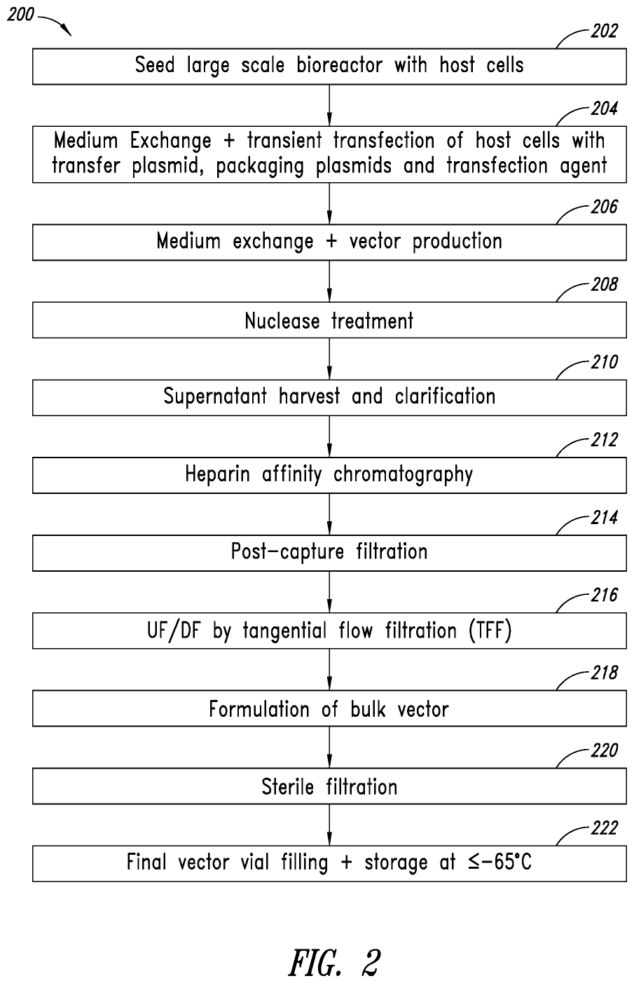

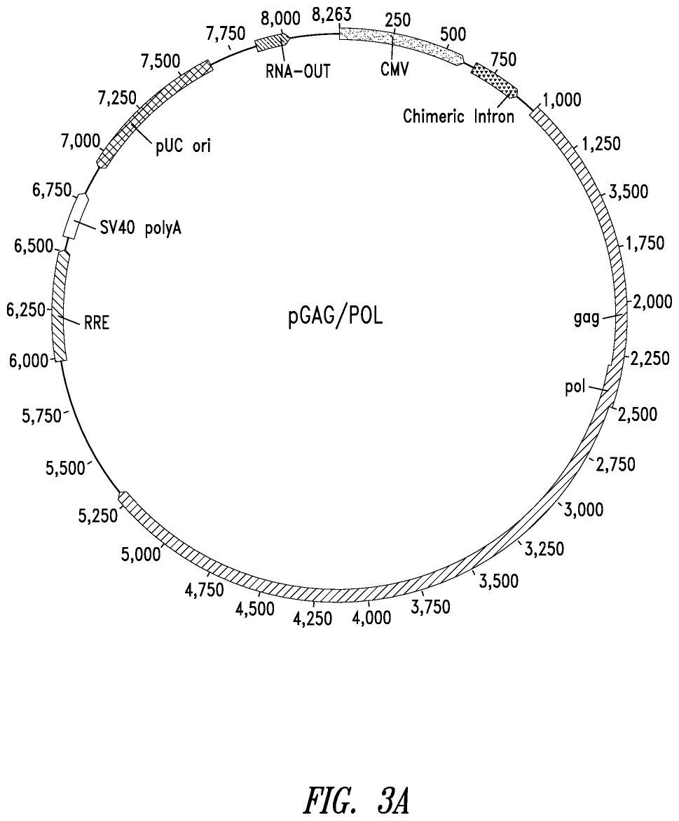

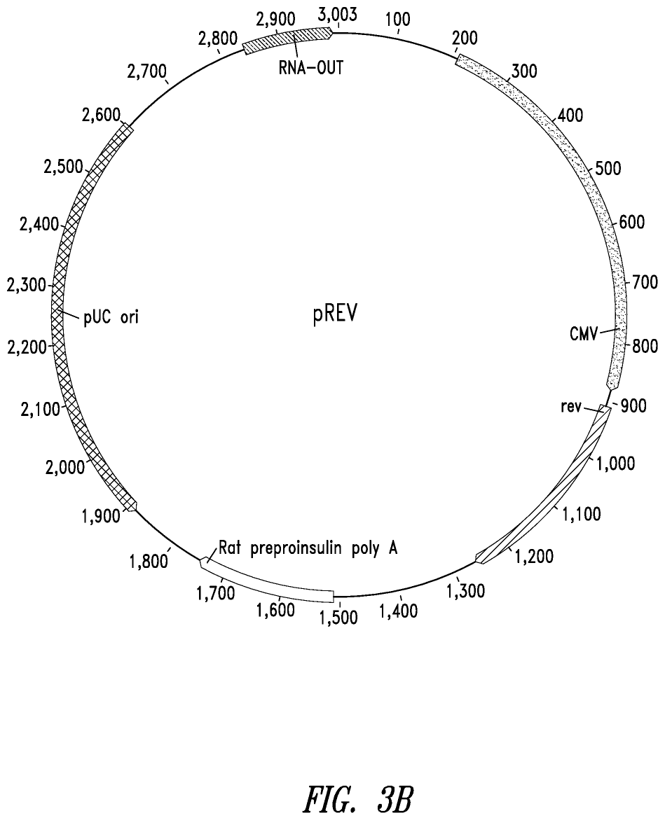

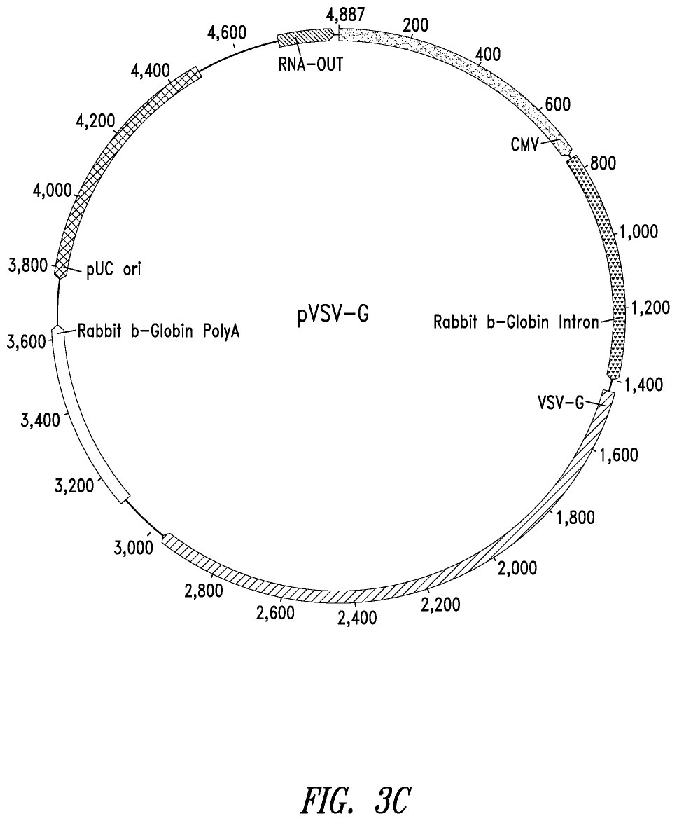

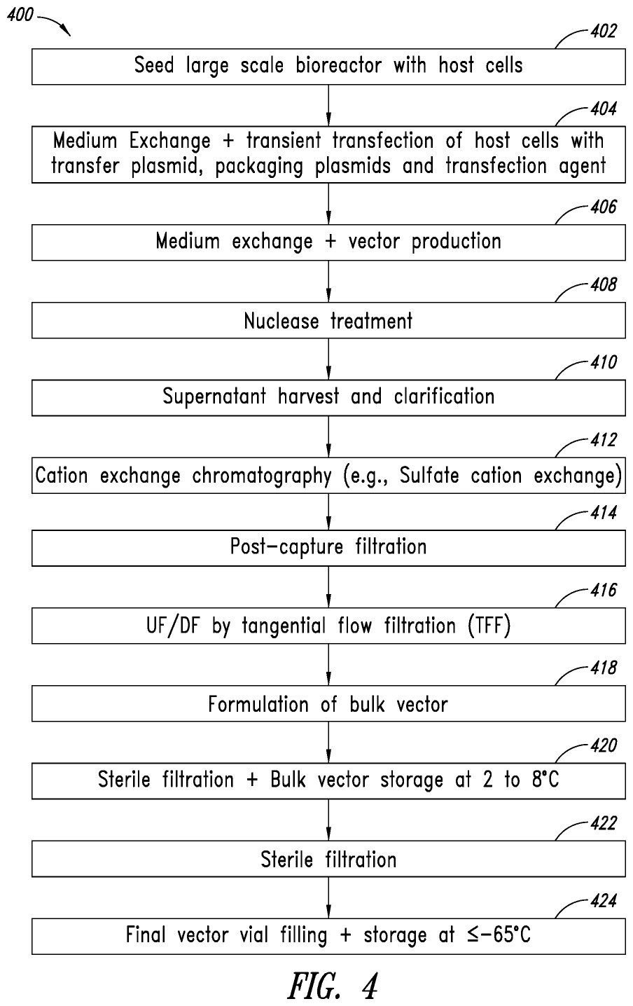

The suspension manufacturing processes described in Examples 1 and 2 (i.e., processes sLVV 2.0 and sLVV 2.5) were compared to an adherent manufacturing process (aLVV). aLVV was produced as described in Gorman et al., Molecular Therapy, Volume 23, Supplement 1, May 2015. sLVV 2.0 was produced as described in Example 1 (see also FIG. 1), while sLVV 2.5 was produced at the 2 L final culture scale (volumes scaled accordingly) and purified until the intermediate bulk unit operation as described in Example 2 (see also FIG. 4). The intermediate bulk step is representative of the final LVV product in terms of residual impurities, and hence will be compared to the final LVV product produced by the 2.0 sLVV process (FIG. 1) and aLVV process. Process yield is determined by infectious titer yield, which is measured by viral transduction of HOS cells. Purity of LVV product is compared based on residual Host Cell Protein (HCP) concentration, which is measured by an enzyme-linked immunosorbent assay (ELISA). Additionally, the relative residual p24 capsid protein concentration versus infectious titer (particle to infectivity ratio) is compared between platforms as a measure of LVV purity. Residual p24 is also measured by ELISA.

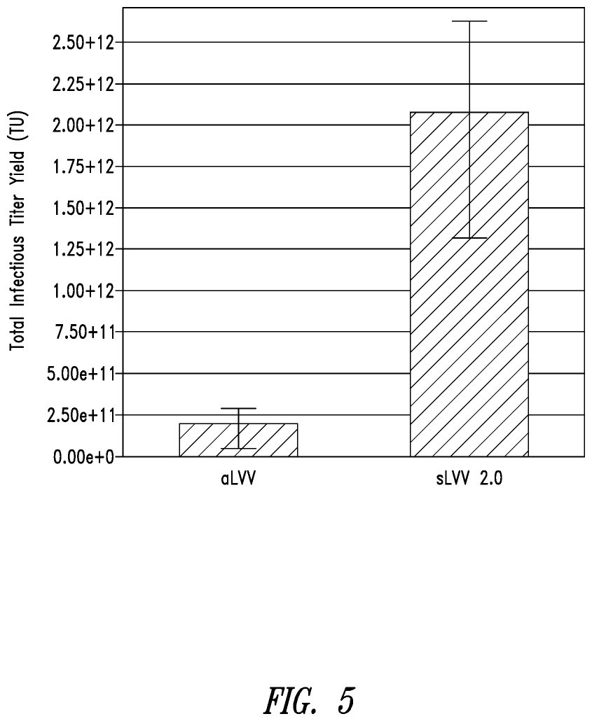

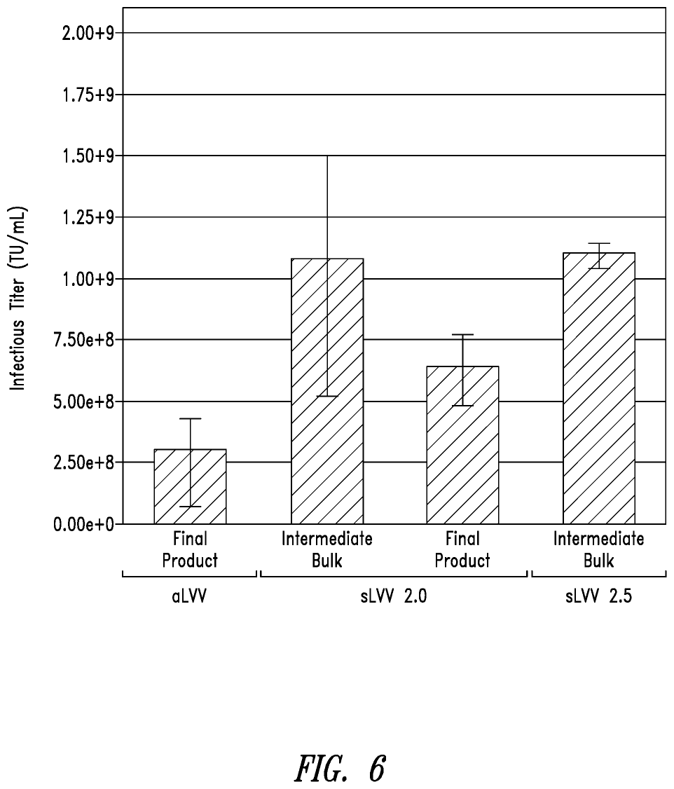

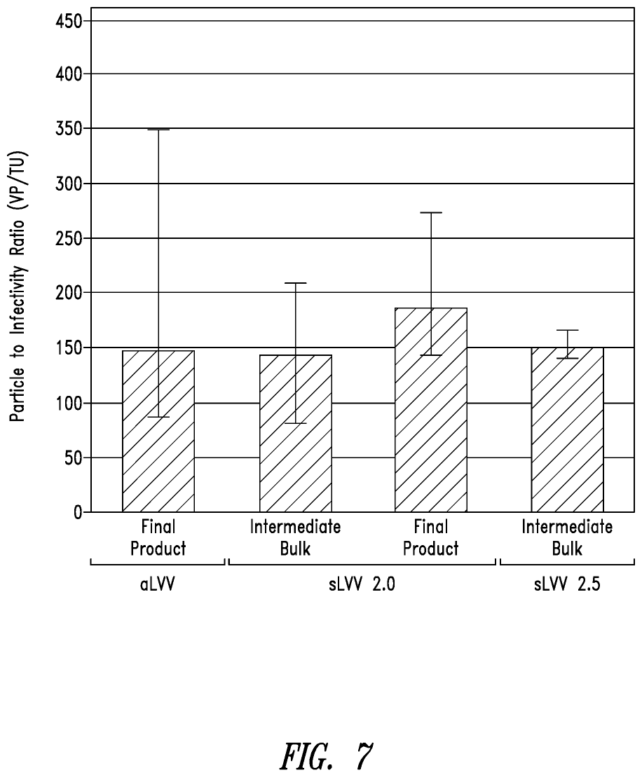

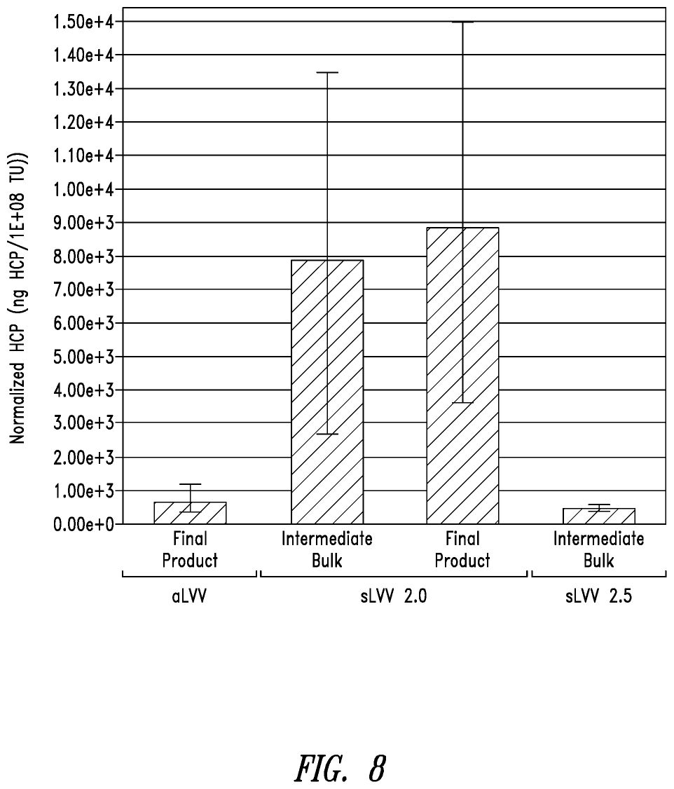

As shown in FIG. 5, the suspension 2.0 sLVV process demonstrates an increase in total infectious titer (TU) by approximately 10-fold compared to the aL VV production process due in part to an increase production cell culture scale (200 L for 2.0 sLVV versus 40 L for aLVV), as well as higher infectious titer concentration as shown in FIG. 6. The 2.5 sLVV process yields similar intermediate bulk titer concentrations to 2.0 sLVV (FIG. 6) and maintains a higher infectious titer than the original aL VV process. Additionally, the 2.0 and 2.5 sLVV processes demonstrate more consistency in particle to infectivity (VP/TU) ratios compared to the aLVV process (see FIG. 7).

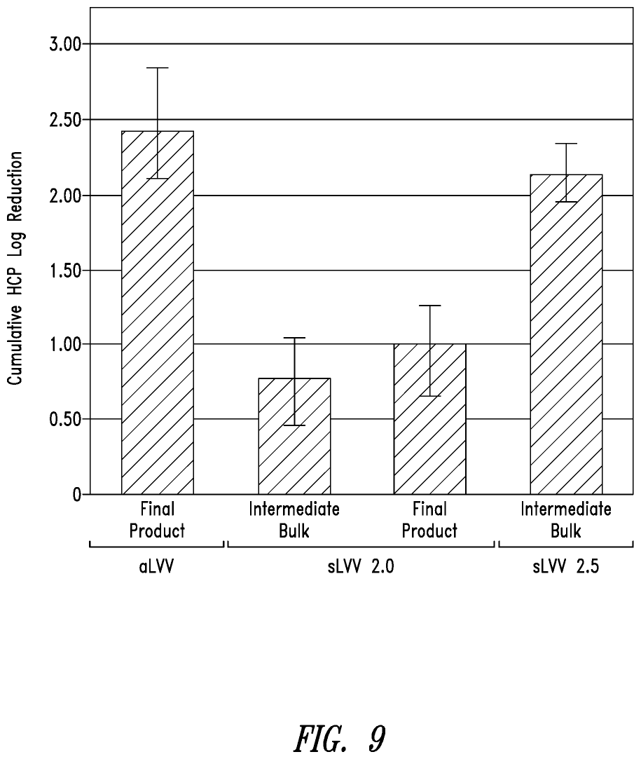

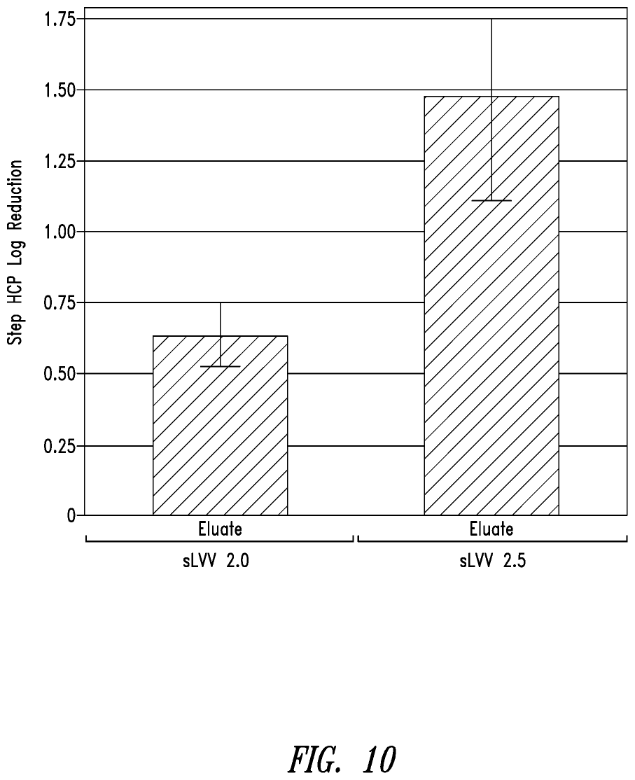

Additionally, the sLVV 2.5 process yields a major improvement in host cell protein (HCP) impurity reduction compared to the sLVV 2.0 process. While the sLVV 2.0 process showed a 10-fold increase in infectious titer compared to the aLVV process, HCP levels also increased throughout the process by greater than 10-fold when normalized to infectious titer. However, the sLVV 2.5 process was able to achieve substantially higher host cell protein reduction from the intermediate bulk LVV, while yielding comparable normalized HCP levels to the aL VV production process (FIG. 8). HCP log reduction value is also similar between the aL VV and sLVV 2.5 processes, which indicates similar impurity removal performance (FIG. 9). Without wishing to be bound by any particular theory, one potential reason for improved HCP removal during the 2.5 sLVV process is attributed to the introduction of the sulfate cation capture chromatography column. As shown in FIG. 10, the heparin affinity binding column achieves an average HCP log reduction of <1.0 logs over the chromatography unit operation step, whereas sulfate column demonstrates ˜1.5 log reduction during chromatography, contributing to an overall increase in HCP reduction for the sLVV 2.5 process of 1.0 logs of HCP (>10-fold decrease in HCP concentration at intermediate bulk).

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A suspension process for producing lentiviral vector (sLVV) comprising:
(a) inoculating a large-scale suspension culture with viable host cells;
(b) transiently transfecting the host cells in the large-scale suspension culture with a mixture comprising lentiviral packaging plasmids, a transfer plasmid, and a transfection agent;
(c) adding an endonuclease to the suspension culture supernatant about 36 hours to about 48 hours post-transfection (after the initiation of transfection);
(d) harvesting and clarifying the suspension culture supernatant using a tandem depth filter and a dual-layer filter;
(e) capturing and concentrating the lentiviral vector from the harvested and clarified suspension culture supernatant using chromatography;
(f) filtering the concentrated lentiviral vector;
(g) ultrafiltering and diafiltering the lentiviral vector using Tangential Flow Filtration (TFF); and
(h) formulating the lentiviral vector to produce a formulated bulk lentiviral vector, and sterile filtering the formulated bulk lentiviral vector.

2. The process of claim 1, wherein the process comprises inoculating a suspension culture of 200 L to 2000 L.

3. The process of claim 1, wherein the process comprises inoculating a suspension culture of 200 L to 1000 L.

4. The process of claim 1, wherein the process comprises inoculating a suspension culture of 200 L to 500 L.

5. The process of claim 1, wherein the process comprises inoculating a suspension culture of 200 L.

6. The process of any one of claims 1 to 5, wherein the large-scale suspension culture is inoculated with about 40.0×10⁸ to about 120.0×10⁸ viable host cells.

7. The process of any one of claims 1 to 6, wherein the host cells are selected from the group consisting of HEK293 cells, HEK293S cells, HEK293T cells adapted for suspension culture (HEK293Ts), HEK293F cells, HEK293FT cells, HEK293FTM cells, and HEK293E cells.

8. The process of any one of claims 1 to 7, wherein the host cells are HEK293Ts cells.

9. The process of any one of claims 1 to 8, wherein the large-scale cell suspension culture comprises host cells cultured in a culture medium.

10. The process of any one of claims 1 to 9, wherein the large-scale cell suspension culture comprises host cells cultured in a culture medium for about 3 days, wherein after the 3 days, the culture medium is exchanged for fresh culture medium.

11. The process of any one of claims 1 to 10, wherein the large-scale cell suspension culture comprises host cells cultured in a culture medium for about 3 days, wherein after the 3 days, the culture medium is exchanged for fresh culture medium using Alternating Tangential Flow Filtration (ATF).

12. The process of any one of claims 9 to 11, wherein the culture medium is a serum-free chemically defined cell culture medium.

13. The process of any one of claims 1 to 12, wherein the host cells are transiently transfected with the mixture comprising a transfection agent selected from the group consisting of: calcium phosphate, cationic lipids, and cationic polymers.

14. The process of any one of claims 1 to 13, wherein the host cells are transiently transfected with the mixture comprising a transfection agent that is a cationic polymer selected from the group consisting of: DEAE-dextran, polybrene, dendrimers, and polyethylenimine (PEI).

15. The process of any one of claims 1 to 14, wherein the host cells are transiently transfected with the mixture comprising a transfection agent that comprises PEI.

16. The process of any one of claims 1 to 15, wherein the transfection agent comprises PEI and the mixture has a ratio of N (NH2 amines in PEI):P (phosphate groups in DNA backbone) of about 5, about 5.5, about 6, about 6.4, about 6.5, about 7, about 7.5, about 8, about 8.5, about 9, about 9.5, or about 10.

17. The process of any one of claims 1 to 16, wherein the transfection agent is added to the suspension culture for about 14 to about 18 hours, optionally wherein the suspension culture is subjected to a culture medium exchange with fresh culture medium using Alternating Tangential Flow Filtration (ATF).

18. The process of any one of claims 1 to 17, wherein the lentiviral packaging plasmids encode lentiviral gag, pol, and rev and a heterologous envelope protein.

19. The process of any one of claims 1 to 18, wherein the lentiviral packaging plasmids encode a heterologous envelope protein selected from the group consisting of a Vesicular stomatitis virus (VSV) envelope protein or variant thereof (e.g., VSV-G), a Cocal virus (COCV) envelope protein or variant thereof, a Maraba virus (MARAV) envelope protein or variant thereof, a Piry virus (PIRYV) envelope protein or variant thereof, a Nipah virus (NiV) envelope protein or variant thereof, a Sendai virus (SeV) envelope protein or variant thereof, a Morbillivirus envelope protein or variant thereof, a Canine distemper (CDV) envelope protein or variant thereof, a Measles virus (MV) envelope protein or variant thereof, a Sindbis virus (SINV) envelope protein or variant thereof, a Gibbon ape leukemia virus (GALV) envelope protein or variant thereof, a Feline endogenous retrovirus (RD114) envelope protein or variant thereof, a Feline leukemia virus (FeLV) envelope protein or variant thereof, a Baboon endogenous retrovirus (BaEV) envelope protein or variant thereof, a Hepatitis B (HBV) envelope protein or variant thereof, a Hepatitis C (HCV) envelope protein or variant thereof, and a Rabis virus (RABV) envelope protein or variant thereof.

20. The process of any one of claims 1 to 19, wherein the transfer plasmid comprises a polynucleotide comprising a packageable lentiviral vector genome.

21. The process of any one of claims 1 to 20, wherein the transfer plasmid comprises a polynucleotide comprising a packageable lentiviral vector genome comprising a left chimeric (5′) lentiviral LTR, the promoter of the 5′ LTR is replaced with a heterologous promoter; a Psi (Ψ) packaging signal; a central polypurine tract/DNA flap (cPPT/FLAP); a retroviral export element (RRE); a promoter operably linked to a polynucleotide of interest; and a right (3′) self-inactivating (SIN) lentiviral LTR.

22. The process of any one of claims 1-21, wherein the endonuclease is derived from Serratia marcescens, optionally wherein the endonuclease is a recombinant NucA endonuclease.

23. The process of any one of claims 1 to 22, wherein the endonuclease has both DNA and RNA cleaving activity.

24. The process of any one of claims 1 to 23, wherein the endonuclease is Benzonase or Denarase.

25. The process of any one of claims 1-24, wherein the endonuclease is added at a concentration of about 60 U/ml or about 30 U/ml.

26. The process of any one of claims 1 to 25, wherein the endonuclease is added to the suspension culture supernatant about 36 hours to about 72 hours post-transfection.

27. The process of any one of claims 1 to 26, wherein the endonuclease is added to the suspension culture supernatant about 36 hours to about 48 hours post-transfection.

28. The process of any one of claims 1 to 27, wherein the endonuclease is added to the suspension culture supernatant about 48 hours post-transfection.

29. The process of any one of claims 1 to 27, wherein the endonuclease is added to the suspension culture supernatant about 44 hours post-transfection.

30. The process of any one of claims 1 to 27, wherein the endonuclease is added to the suspension culture supernatant about 40 hours after transfection.

31. The process of any one of claims 1 to 27, wherein the endonuclease is added to the suspension culture supernatant about 36 hours after transfection.

32. The process of any one of claims 1 to 31, wherein the endonuclease is added to the culture for about 1 to about 2 hours.

33. The process of any one of claims 1 to 32, wherein the harvesting and clarifying step comprises filtering the suspension culture supernatant through the tandem depth filter retains contaminants of at least about 40 μm or at least about 60 μm and a dual-layer filter comprising a pre-filter pore size of about 0.45 μm to about 0.8 μm and a final filter pore size of about 0.22 μm to about 0.45 μm.

34. The process of claim 33, wherein the clarified suspension culture is adjusted to about pH 7.0 or about pH 7.2, optionally with 1M HEPES.

35. The process of any one of claims 1 to 34, wherein the lentiviral vector is captured and concentrated from the harvested and clarified suspension culture supernatant using affinity chromatography or cation exchange chromatography.

36. The process of claim 35, wherein the supernatant is passed over an affinity chromatography column or cation exchange chromatography column.

37. The process of claim 35 or 36, wherein the affinity chromatography is a heparin affinity chromatography.

38. The process of claim 35 or 36, wherein the cation exchange chromatography is a sulfate cation exchange chromatography.

39. The process of claim 38, wherein the sulfate cation exchange chromatography comprises a column having bead size of about 45 μm and/or a mean pore size of about 100 nm.

40. The process of any one of claims 1 to 39, wherein a wash buffer comprising about 50 mM HEPES, about 100 mM NaCl, pH 7 is pumped over the chromatography column.

41. The process of any one of claims 1 to 39, wherein an elution buffer comprising about 50 mM HEPES, about 400 mM NaCl, pH 8 is pumped over the chromatography column.

42. The process of any one of claims 1 to 39, wherein a wash buffer comprising about 50 mM HEPES, about 300 mM NaCl, pH 7.2 is passed over the chromatography column.

43. The process of any one of claims 1 to 39, wherein an elution buffer comprising about 50 mM HEPES, about 1 M NaCl, pH 7.5 is passed over the chromatography column.

44. The process of any one of claims 1 to 43, wherein the filtering step (f) comprises filtering the concentrated lentiviral vector through a dual-layer filter comprising a pre-filter pore size of about 0.45 μm to about 0.8 μm and a final filter pore size of about 0.2 μm to about 0.45 μm.

45. The process of any one of claims 1 to 44, wherein the lentiviral vector is ultrafiltered and diafiltered using a hollow fiber Tangential Flow Filtration (TFF) filter comprising an about 100 kDa to about 500 kDa pore size or molecular weight cutoff.

46. The process of claim 45, wherein the hollow fiber TFF filter comprises a pore size or molecular weight cutoff of about 100 kDa.

47. The process of claim 45, wherein the hollow fiber TFF filter comprises a pore size or molecular weight cutoff of about 300 kDa.

48. The process of claim 45, wherein the hollow fiber TFF filter comprises a pore size or molecular weight cutoff of about 500 kDa.

49. The process of any one of claims 45 to 48, wherein the lentiviral vector is diafiltered into diafiltration buffer, optionally wherein the diafiltration buffer is about 50 mM HEPES, about 100 mM NaCl, pH 7.50.

50. The process of any one of claims 45 to 48, wherein the lentiviral vector is diafiltered into diafiltration buffer, optionally wherein the diafiltration buffer is about 50 mM HEPES, pH 7.0.

51. The process of any one of claims 45 to 48, wherein the lentiviral vector is diafiltered into diafiltration buffer, optionally wherein the diafiltration buffer is about 50 mM L-His, pH 7.0.

52. The process of any one of claims 1 to 51, wherein the lentiviral vector is formulated 1:1 in 2× Stem Cell Growth Medium (SCGM) to produce the formulated bulk lentiviral vector.

53. The process of any one of claims 1 to 51, wherein the lentiviral vector is formulated 1:1 in a buffer comprising HEPES and Sucrose, optionally wherein the buffer further comprises L-proline, poloxamer 188, or NaCl.

54. The process of any one of claims 1 to 51, wherein the lentiviral vector is formulated 1:1 in a buffer comprising about 5 mM HEPES (pH 7.0), about 146 mM Sucrose, and about 100 mM L-proline.

55. The process of any one of claims 1 to 51, wherein the lentiviral vector is formulated 1:1 in a buffer comprising about 5 mM HEPES (pH 7.0), about 146 mM Sucrose, about 100 mM L-proline, and about 0.2 to about 2.0 poloxamer 188.

56. The process of any one of claims 1 to 51, wherein the lentiviral vector is formulated 1:1 in a buffer comprising about 5 mM HEPES (pH 7.0), about 146 mM Sucrose, about 100 mM L-proline, and 150 mM NaCl.

57. The process of any one of claims 1 to 51, wherein the lentiviral vector is formulated 1:1 in a buffer comprising about 5 mM HEPES (pH 7.0), about 146 mM Sucrose, about 100 mM L-proline, 150 mM NaCl, and about 0.2 to about 2.0 mg/ml poloxamer 188.

58. The process of any one of claims 1 to 51, wherein the lentiviral vector is formulated 1:1 in a buffer comprising about 5 mM HEPES (pH 7.0), about 146 mM Sucrose, about 150 mM NaCl, and about 0.2 to about 2.0 mg/ml poloxamer 188.

59. The process of any one of claims 1 to 51, wherein the lentiviral vector is formulated 1:1 in a buffer comprising L-Histidine, Sucrose, and L-proline.

60. The process of any one of claims 1 to 51, wherein the lentiviral vector is formulated 1:1 in a buffer comprising about 5 mM L-Histidine, about 146 mM Sucrose, and about 100 mM L-proline, optionally wherein the formulation further comprises about 0.2 to about 2.0 mg/mL poloxamer 188.

61. The process of any one of claims 1 to 60, wherein the formulated bulk lentiviral vector is sterile filtered through a 0.22 μm filter, optionally comprising a 0.45 μm pre-filter.

62. The process of any one of claims 1 to 61, wherein the process further comprises performing a fill finish of the formulated bulk lentiviral vector to produce a final lentiviral vector, and freezing the final lentiviral vector.

63. The process of any one of claims 1 to 61, wherein the process further comprises freezing the formulated bulk lentiviral vector.

64. The process of claim 63, wherein the process further comprises thawing the formulated bulk lentiviral vector, sterile filtering the formulated bulk lentiviral vector, performing a fill finish of the formulated bulk lentiviral vector to produce a final lentiviral vector, and freezing the final lentiviral vector.

65. The process of any one of claims 62 to 64, wherein the final lentiviral vector is frozen at ≤−65° C.

66. A method for producing suspension lentiviral vector comprising:
(a) inoculating a P0 suspension culture comprising about 50 mL of culture medium with about 10.0×10⁶ to about 15.0×10⁶ viable HEK293Ts cells;
(b) inoculating a P1 suspension culture comprising about 100 mL of culture medium with about 30.0×10⁶ to about 70.0×10⁶ viable HEK293Ts cells obtained from the P0 suspension culture;
(c) inoculating three P2 suspension cultures each comprising about 200 mL of culture medium with about 11.0×10⁷ to about 19.0×10⁷ viable HEK293Ts cells obtained from the P1 suspension culture;
(d) inoculating three P3 suspension cultures each comprising about 1.0 L of culture medium with about 55.0×10⁷ to about 95.0×10⁷ viable HEK293Ts cells obtained from the pooled P2 suspension cultures;
(e) inoculating a P4 suspension culture comprising about 20.0 L of culture medium with about 40.0×10⁸ to about 120.0×10⁸ viable HEK293Ts cells obtained from the pooled P3 suspension cultures;
(f) inoculating a P5 suspension culture comprising about 200.0 L of culture medium with about 40.0×10⁸ to about 120.0×10⁸ viable HEK293Ts cells obtained from the P4 suspension culture;
(g) culturing the P5 suspension culture for about 3 days and exchanging the culture medium of the P5 suspension culture with about 190.0 L of fresh culture medium using Alternating Tangential Flow Filtration (ATF);
(h) transfecting the P5 suspension culture, after the culture medium exchange, to produce a lentiviral vector, said transfecting step comprising adding about 10.0 L of culture medium comprising a transfer plasmid and plasmid DNAs encoding, gag, pol, rev, VSV-g, complexed with polyethyleneimine (PEI);
(i) exchanging the culture medium of the P5 suspension culture, post-transfection, with about 200.0 L of fresh culture medium using ATF;
(j) about 36 hours to about 48 hours after post-transfection, treating the P5 suspension culture with an endonuclease for about 1 hour to about 2 hours;
(k) harvesting and clarifying the P5 suspension culture supernatant using a tandem depth filter that retains particles of 60 μm or greater, and a dual-layer filter comprising 0.8 μm and 0.45 μm pore sizes;
(l) capturing and concentrating the lentiviral vector from the harvested and clarified P5 suspension culture supernatant comprising heparin chromatography or sulfate cation exchange chromatography;
(m) filtering the concentrated lentiviral vector using a dual-layer filter comprising 0.8 μm and 0.45 μm pore sizes;
(n) ultrafiltering the lentiviral vector using Tangential Flow Filtration (TFF) to further concentrate the lentiviral vector and diafiltering the lentiviral vector into diafiltration buffer, thereby producing bulk lentiviral vector; and
(o) formulating the lentiviral vector to produce a formulated bulk lentiviral vector.

67. The method of claim 66, wherein the culture medium is a serum-free chemically defined cell culture medium.

68. The method of claim 66 or claim 67, wherein the ratio of N (NH2 amines in PEI):P (phosphate groups in DNA backbone) for the PEI/DNA mixture is about 5, about 5.5, about 6, about 6.4, about 6.5, about 7, about 7.5, about 8, about 8.5, about 9, about 9.5, or about 10.

69. The process of any one of claims 66 to 68, wherein the PEI is added to the suspension culture for about 14 to about 18 hours, optionally wherein the suspension culture is subjected to a culture medium exchange with fresh culture medium using Alternating Tangential Flow Filtration (ATF).

70. The method of any one of claims 66 to 69, wherein the transfer plasmid comprises a polynucleotide comprising a packageable lentiviral vector genome.

71. The method of any one of claims 66 to 70, wherein the transfer plasmid comprises a polynucleotide comprising a packageable lentiviral vector genome comprising a left chimeric (5′) lentiviral LTR, the promoter of the 5′ LTR is replaced with a heterologous promoter; a Psi (Ψ) packaging signal; a central polypurine tract/DNA flap (cPPT/FLAP); a retroviral export element (RRE); a promoter operably linked to a polynucleotide of interest; and a right (3′) self-inactivating (SIN) lentiviral LTR.

72. The process of any one of claims 66 to 71, wherein the endonuclease is derived from Serratia marcescens.

73. The process of any one of claims 66 to 72, wherein the endonuclease has both DNase and RNase activity.

74. The method of any one of claims 66 to 73, wherein the endonuclease is Benzonase or Denarase.

75. The process of any one of claims 66 to 74, wherein the endonuclease is added at a concentration of about 60 U/ml or about 30 U/ml.

76. The process of claims 66 to 75, wherein the clarified suspension culture is adjusted to about pH 7.0 or about pH 7.2, optionally with 1M HEPES.

77. The process of claims 66 to 76, wherein the sulfate cation exchange chromatography comprises a column having bead size of about 45 μm and/or a mean pore size of about 100 nm.

78. The process of any one of claims 66 to 76, wherein a wash buffer comprising about 50 mM HEPES, about 100 mM NaCl, pH 7 is pumped over the heparin chromatography column.

79. The process of any one of claims 66 to 76, wherein an elution buffer comprising about 50 mM HEPES, about 400 mM NaCl, pH 8 is pumped over the heparin chromatography column.

80. The process of any one of claims 66 to 76, wherein a wash buffer comprising about 50 mM HEPES, about 300 mM NaCl, pH 7.2 is pumped over the cation exchange chromatography column.

81. The process of any one of claims 66 to 76, wherein an elution buffer comprising about 50 mM HEPES, about 1 M NaCl, pH 7.5 is pumped over the cation exchange chromatography.

82. The method of any one of claims 66 to 81, wherein the lentiviral vector is ultrafiltered and diafiltered into diafiltration buffer using a hollow fiber TFF filter comprising a pore size or molecular weight cutoff of about 100 kDa, about 300 kDa or about 500 kDa.

83. The method of any one of claims 66 to 82, wherein the diafiltration buffer is about 50 mM HEPES, about 100 mM NaCl, pH 7.50.

84. The process of any one of claims 66 to 82, wherein the diafiltration buffer is about 50 mM HEPES, pH 7.0.

85. The process of any one of claims 66 to 82, wherein the lentiviral vector is diafiltered into diafiltration buffer, optionally wherein the diafiltration buffer is about 50 mM L-Histidine, pH 7.0.

86. The process of any one of claims 66 to 84, wherein the lentiviral vector is formulated 1:1 in 2× Stem Cell Growth Medium (SCGM) to produce the formulated bulk lentiviral vector.

87. The process of any one of claims 66 to 84, wherein the lentiviral vector is formulated 1:1 in a buffer comprising HEPES and Sucrose, optionally wherein the buffer further comprises L-proline, poloxamer 188, or NaCl.

88. The process of any one of claims 66 to 84, wherein the lentiviral vector is formulated 1:1 in a buffer comprising about 5 mM HEPES (pH 7.0), about 146 mM Sucrose, and about 100 mM L-proline.

89. The process of any one of claims 66 to 84, wherein the lentiviral vector is formulated 1:1 in a buffer comprising about 5 mM HEPES (pH 7.0), about 146 mM Sucrose, about 100 mM L-proline, and about 0.2 to about 2.0 mg/ml poloxamer 188.

90. The process of any one of claims 66 to 84, wherein the lentiviral vector is formulated 1:1 in a buffer comprising about 5 mM HEPES (pH 7.0), about 146 mM Sucrose, about 100 mM L-proline, and about 150 mM NaCl.

91. The process of any one of claims 66 to 84, wherein the lentiviral vector is formulated 1:1 in a buffer comprising about 5 mM HEPES (pH 7.0), about 146 mM Sucrose, about 100 mM L-proline, about 150 mM NaCl, and about 0.2 to about 2.0 mg/ml poloxamer 188.

92. The process of any one of claims 66 to 84, wherein the lentiviral vector is formulated 1:1 in a buffer comprising about 5 mM HEPES (pH 7.0), about 146 mM Sucrose, about 150 mM NaCl, and about 0.2 to about 2.0 mg/ml poloxamer 188.

93. The process of any one of claims 66 to 84, wherein the lentiviral vector is formulated 1:1 in a buffer comprising L-Histidine, Sucrose, and L-proline.

94. The process of any one of claims 66 to 84, wherein the lentiviral vector is formulated 1:1 in a buffer comprising about 5 mM L-Histidine, about 146 mM Sucrose, and about 100 mM L-proline, optionally wherein the formulation further comprises about 0.2 to about 2.0 mg/ml poloxamer 188.

95. The method of any one of claims 66 to 94, wherein the method further comprises performing a fill finish of the formulated bulk lentiviral vector to produce a final lentiviral vector, and freezing the final lentiviral vector.

96. The method of any one of claims 66 to 94, wherein the method further comprises freezing the formulated bulk lentiviral vector.

97. The method of claim 96, wherein the process further comprises thawing the formulated bulk lentiviral vector, sterile filtering the formulated bulk lentiviral vector, performing a fill finish of the formulated bulk lentiviral vector to produce a final lentiviral vector, and freezing the final lentiviral vector.

98. The process of any one of claims 95 to 97, wherein the final lentiviral vector is frozen at ≤−65° C.

99. A method of reducing host cell protein (HCP) from a suspension process for producing viral vector comprising:
(a) providing a harvested and clarified suspension culture supernatant comprising a viral vector (e.g., a viral vector manufactured according to any one of the preceding claims);
(b) capturing and concentrating the viral vector from the harvested and clarified suspension culture supernatant using cation exchange chromatography;
(c) filtering the concentrated viral vector;
(d) ultrafiltering and diafiltering the viral vector using Tangential Flow Filtration (TFF); and
(e) formulating the viral vector to produce a formulated bulk viral vector, and sterile filtering the formulated bulk viral vector.

100. The process of claim 99, wherein the viral vector is a lentiviral vector.

101. The process of claim 99 or claim 100, wherein the viral vector is pseudotyped with a heterologous envelope protein selected from the group consisting of a Vesicular stomatitis virus (VSV) envelope protein or variant thereof (e.g., VSV-G), a Cocal virus (COCV) envelope protein or variant thereof, a Maraba virus (MARAV) envelope protein or variant thereof, a Piry virus (PIRYV) envelope protein or variant thereof, a Nipah virus (NiV) envelope protein or variant thereof, a Sendai virus (SeV) envelope protein or variant thereof, a Morbillivirus envelope protein or variant thereof, a Canine distemper (CDV) envelope protein or variant thereof, a Measles virus (MV) envelope protein or variant thereof, a Sindbis virus (SINV) envelope protein or variant thereof, a Gibbon ape leukemia virus (GALV) envelope protein or variant thereof, a Feline endogenous retrovirus (RD114) envelope protein or variant thereof, a Feline leukemia virus (FeLV) envelope protein or variant thereof, a Baboon endogenous retrovirus (BaEV) envelope protein or variant thereof, a Hepatitis B (HBV) envelope protein or variant thereof, a Hepatitis C (HCV) envelope protein or variant thereof, and a Rabis virus (RABV) envelope protein or variant thereof.

102. The process of any one of claims 99-101 the viral vector is pseudotyped with a heterologous envelope protein consisting of a Vesicular stomatitis virus (VSV) envelope protein or variant thereof (e.g., VSV-G).

103. The process of any one of claims 99-102, wherein the harvesting and clarifying step comprises filtering the suspension culture supernatant through the tandem depth filter retains contaminants of at least about 40 μm or at least about 60 μm and a dual-layer filter comprising a pre-filter pore size of about 0.45 μm to about 0.8 μm and a final filter pore size of about 0.22 μm to about 0.45 μm.

104. The process of claim 103, wherein the clarified suspension culture is adjusted to about pH 7.0 or about pH 7.2, optionally with 1M HEPES.

105. The process of any one of claims 99-104, wherein the supernatant is passed over the cation exchange chromatography column.

106. The process of claim 105, wherein the cation exchange chromatography is a sulfate cation exchange chromatography.

107. The process of claim 106, wherein the sulfate cation exchange chromatography comprises a column having bead size of about 45 μm and/or a mean pore size of about 100 nm.

108. The process of any one of claims 99 to 107, wherein a wash buffer comprising about 50 mM HEPES, about 300 mM NaCl, pH 7.2 is passed over the chromatography column.

109. The process of any one of claims 99 to 107, wherein an elution buffer comprising about 50 mM HEPES, about 1 M NaCl, pH 7.5 is passed over the chromatography column.

110. The process of any one of claims 99 to 109, wherein the filtering step (c) comprises filtering the concentrated viral vector through a dual-layer filter comprising a pre-filter pore size of about 0.45 μm to about 0.8 μm and a final filter pore size of about 0.2 μm to about 0.45 μm.

111. The process of any one of claims 99 to 110, wherein the viral vector is ultrafiltered and diafiltered using a hollow fiber Tangential Flow Filtration (TFF) filter comprising an about 100 kDa to about 500 kDa pore size or molecular weight cutoff.

112. The process of claim 111, wherein the hollow fiber TFF filter comprises a pore size or molecular weight cutoff of about 100 kDa.

113. The process of claim 111, wherein the hollow fiber TFF filter comprises a pore size or molecular weight cutoff of about 300 kDa.

114. The process of claim 111, wherein the hollow fiber TFF filter comprises a pore size or molecular weight cutoff of about 500 kDa.

115. The process of any one of claims 111 to 114, wherein the viral vector is diafiltered into diafiltration buffer, optionally wherein the diafiltration buffer is about about 50 mM HEPES, pH 7.0.

116. The process of any one of claims 99 to 115, wherein the viral vector is formulated 1:1 in a buffer comprising HEPES and Sucrose, optionally wherein the buffer further comprises L-proline, poloxamer 188, or NaCl.

117. The process of any one of claims 99 to 115, wherein the viral vector is formulated 1:1 in a buffer comprising about 5 mM HEPES (pH 7.0), about 146 mM Sucrose, and about 100 mM L-proline.

118. The process of any one of claims 99 to 115, wherein the viral vector is formulated 1:1 in a buffer comprising about 5 mM HEPES (pH 7.0), about 146 mM Sucrose, about 100 mM L-proline, and about 0.2 to about 2.0 poloxamer 188.

119. The process of any one of claims 99 to 115, wherein the viral vector is formulated 1:1 in a buffer comprising about 5 mM HEPES (pH 7.0), about 146 mM Sucrose, about 100 mM L-proline, and about 150 mM NaCl.

120. The process of any one of claims 99 to 115, wherein the viral vector is formulated 1:1 in a buffer comprising about 5 mM HEPES (pH 7.0), about 146 mM Sucrose, about 100 mM L-proline, about 150 mM NaCl, and about 0.2 to about 2.0 mg/ml poloxamer 188.

121. The process of any one of claims 99 to 115, wherein the viral vector is formulated 1:1 in a buffer comprising about 5 mM HEPES (pH 7.0), about 146 mM Sucrose, about 150 mM NaCl, and about 0.2 to about 2.0 mg/ml poloxamer 188.

122. The process of any one of claims 99 to 121, wherein the formulated bulk viral vector is sterile filtered through a 0.22 μm filter, optionally comprising a 0.45 μm pre-filter.

123. The process of any one of claims 99 to 121, wherein the process further comprises performing a fill finish of the formulated bulk viral vector to produce a final viral vector, and freezing the final viral vector.

124. The process of claim 123, wherein the process further comprises freezing the formulated bulk viral vector.

125. The process of any one of claims claims 122 to 124, wherein the process further comprises thawing the formulated bulk viral vector, sterile filtering the formulated bulk viral vector, performing a fill finish of the formulated bulk viral vector to produce a final viral vector, and freezing the final viral vector.

126. The process of any one of claims 123 to 125, wherein the final viral vector is frozen at ≤−65° C
